(19) United States
(12) Patent Application Publication
(10) Pub. No.: US 2026/0294858 A1
(43) Pub. Date: Oct. 1, 2026

(54) LEVOTHYROXINE DISPERSIONS

(71) Applicants: MAIA Pharmaceuticals, Inc., Princeton, NJ (US); MAIARESEARCH INDIA PRIVATE LIMITED, New Delhi (IN)

(72) Inventors: Srikanth SUNDARAM, Somerset, NJ (US); Riyaz Ahammad SHAIK, Vikaspuri, Hyderabad Telangana (IN); Lakshmikantha Reddy PONNAPUREDDY, Kadapa, Andhra Pradesh (IN)

(21) Appl. No.: 19/477,693

(22) PCT Filed: Apr. 27, 2024
(86) PCT No.: PCT/IB2024/054111

§ 371 Date: Oct. 22, 2025

(30) Foreign Application Priority Data
Apr. 28, 2023 (IN) 202311030765

(51) Int. Cl. A61K 31/198; A61K 9/00; A61K 9/10; A61K 9/19; A61K 47/02; A61K 47/10; A61K 47/20

(52) U.S. Cl. CPC A61K 31/198; A61K 9/0019; A61K 9/10; A61K 9/19; A61K 47/02; A61K 47/10; A61K 47/20

(57) ABSTRACT

The present invention relates to pharmaceutical dispersions comprising levothyroxine or its pharmaceutically acceptable salts which is primarily dispersed (e.g., primarily suspended) in a pharmaceutically acceptable carrier. These levothyroxine pharmaceutical dispersions can be long-acting and are useful in treating and/or preventing myxedema coma, hypothyroidism, and/or other thyroid disorders.

### FIELD OF THE INVENTION

The present invention includes parenteral pharmaceutical dispersions (such as long-acting suspensions) comprising a therapeutically effective amount of levothyroxine or a pharmaceutically acceptable salt thereof primarily dispersed in a pharmaceutically acceptable carrier, methods of preparation thereof, and methods of treating myxedema coma, hypothyroidism, and/or other thyroid disorders by parenterally administering the pharmaceutical dispersions described herein.

### BACKGROUND OF THE INVENTION

Levothyroxine sodium injectable products (liquid solution for intravenous injection, and powder for injection reconstituted to solution prior to administration) are approved in the U.S. for treating myxedema coma. The recommended dosage regimen involves intravenous administration of a loading dose of 300 mcg to 500 mcg followed by a maintenance dose of 50 mcg to 100 mcg daily until the patient can tolerate oral therapy. These products are intended to be administered in an acute or hospital setting. There are no approved levothyroxine sodium injectable products in the U.S. for the treatment of hypothyroidism or other thyroid disorders.

U.S. Pat. No. 9,782,376 describes a liquid formulation comprising levothyroxine or its pharmaceutically acceptable salt thereof, tromethamine, sodium iodide and water. The pH of the formulation is about 9.0 to about 11.5.

U.S. Pat. No. 11,154,498 describes liquid formulations comprising levothyroxine sodium, buffering agents, stabilizing agents and/or solubilizing agents, one or more solvents, and optionally one or more pharmaceutically acceptable excipients selected from pH adjusting agents and anti-oxidants.

PCT publication WO 2023/057088 discloses sustained release levothyroxine formulations based on poly(D,L-lactide-co-glycolide) microparticles.

Levothyroxine sodium in the form of a capsule, tablet and solution are approved for oral administration for treating hypothyroidism and pituitary thyrotropin (thyroid-stimulating hormone, TSH) suppression. The oral administration involves administration of levothyroxine, as a single daily-dose, on an empty stomach, one-half to one hour before breakfast. Further, it should be administered at least 4 hours before or after drugs that are known to interfere with absorption of levothyroxine. Many classes of drugs are known to affect levothyroxine pharmacokinetics and metabolism. Further, dose adjustments may be needed when regularly administering within one hour of certain foods that may affect absorption. See the prescribing information for Synthroid®. As a result, daily oral administration results in highly variable bioavailability dependent on factors such as time of administration, dietary habits, and other drugs that the patient requires. Daily oral administration is also highly inconvenient, resulting in poor patient compliance, which ultimately results in serious consequences, including poor clinical outcomes, higher hospitalization rates, and increased healthcare costs.

There is therefore a need in the art to provide an effective treatment option by providing a convenient dosage regimen of levothyroxine that results in consistent absorption for treating hypothyroidism, myxedema coma and other thyroid disorders. The present invention addresses the drawbacks associated with previous levothyroxine formulations.

### SUMMARY OF THE INVENTION

In one embodiment, the present invention provides a pharmaceutical dispersion comprising (a) a therapeutically effective amount of levothyroxine or its pharmaceutically acceptable salt (e.g., levothyroxine sodium), (b) one or more pharmaceutically acceptable excipients and (c) one or more pharmaceutically acceptable carriers, wherein the levothyroxine or its pharmaceutically acceptable salt is primarily dispersed in the one or more pharmaceutically acceptable carriers.

Another embodiment is a stable pharmaceutical suspension comprising (a) a therapeutically effective amount of levothyroxine or its pharmaceutically acceptable salt, (b) one or more pharmaceutically acceptable excipients and (c) one or more pharmaceutically acceptable carriers, wherein the levothyroxine or its pharmaceutically acceptable salt is primarily suspended in the one or more pharmaceutically acceptable carriers.

Yet another embodiment is an aqueous suspension comprising a therapeutically effective amount of levothyroxine or its pharmaceutically acceptable salt primarily suspended in an aqueous carrier.

In certain embodiments, the pharmaceutical dispersions (including the suspensions) described herein are suitable for parenteral administration, preferably intravenous (IV), intramuscular (IM) and/or subcutaneous (SC) administration.

In one embodiment of any pharmaceutical dispersion or suspension described herein, the particle size of the levothyroxine or its pharmaceutically acceptable salt is suitable for parenteral administration, preferably intravenous, subcutaneous and/or intramuscular administration. These particles of levothyroxine may contain one or more pharmaceutically acceptable excipients. The particle size of levothyroxine or its pharmaceutically acceptable salts in such dispersions can vary depending on the intended duration of release and/or therapeutic activity of the long-acting dispersion. Further, in another embodiment, the particle size is suitable for intramuscular and subcutaneous and intravenous administration. In one embodiment, the median particle size of the levothyroxine or its salt is from about 50 nm to about 100 μm, such as about 100 nm to about 75 μm, such as from about 20 or 40 μm to about 100 μm, from about 100 nm to about 2500 nm, from about 100 to about 500 nm, or about 1500 to about 2500 nm.

In one embodiment of any pharmaceutical dispersion or suspension described herein, the levothyroxine or its pharmaceutically acceptable salt is primarily suspended in a pharmaceutically acceptable carrier.

In one embodiment of any pharmaceutical dispersion or suspension described herein, the pH of the dispersion or suspension is suitable for parenteral administration, for example, from about 3 to about 11.5, such as about 4 to about 10, about 5 to about 9, or about 5 to about 8.

In one embodiment of any pharmaceutical dispersion or suspension described herein, the osmolality of the dispersion or suspension is suitable for parenteral administration, for example, less than about 600 mOsmol/kg, about 200 to about 500 mOsmol/kg, about 250 to about 400 mOsmol/kg, or about 300 mOsmol/kg.

In one embodiment of any pharmaceutical dispersion or suspension described herein, the concentration of levothyroxine or a salt thereof is about 20 mcg/mL to about 10 mg/mL, about 100 mcg/mL to about 7.5 mg/mL, about 500 mcg/mL to about 5 mg/mL, about 200 to about 1000 mcg/mL, or about 1 mg/mL to about 3 mg/mL.

In one embodiment of any pharmaceutical dispersion or suspension described herein, the volume of administration is suitable for intravenous, intramuscular and/or subcutaneous administration, such as, for example, less than 10 mL, less than 5 mL, less than about 3 mL, or less than 1.5 mL. In one embodiment, the volume of administration is at least 0.1 mL. For example, the volume of administration is from about 0.1 to about 10 mL. The volume of pharmaceutical dispersion or suspension may be contained in a vial or syringe.

In another embodiment, any of the pharmaceutical dispersions or suspensions described herein is provided in lyophilized powder form, e.g., for reconstitution for IV, SC and IM administration. The lyophilized form may be presented in a vial, or dual chamber syringe or vial or cartridge (lyophilized powder in one chamber and diluent in another chamber). The lyophilized powder and a diluent for reconstitution may be supplied in separate vials. The present invention is also provided as a kit containing the lyophilized powder in vial and the diluent in vial or prefilled syringe (PFS).

In another embodiment, the pharmaceutical compositions described herein may be prepared by mixing (i) a liquid solution containing levothyroxine or its pharmaceutically acceptable salt, which optionally contains one or more excipients, with (ii) a pharmaceutically acceptable carrier, which optionally contains one or more excipients, to form the composition, where the levothyroxine or its pharmaceutically acceptable salt is primarily dispersed, preferably suspended, in the pharmaceutically acceptable carrier. The solution of levothyroxine or its pharmaceutically acceptable salt and the pharmaceutically acceptable carrier for preparation of the final composition may be supplied in separate vials. One embodiment is a dual chamber syringe or vial or cartridge (e.g., dual chamber vial or dual chamber syringe) wherein the solution of levothyroxine or its pharmaceutically acceptable salt is in one chamber and the pharmaceutically acceptable carrier is in a second chamber. The dual chamber syringe may include a system for mixing the contents of the two chambers. The present invention also provides a kit containing the solution of levothyroxine or its pharmaceutically acceptable salt in a vial and the pharmaceutically acceptable carrier in a vial or a prefilled syringe (PFS).

In another embodiment of any pharmaceutical dispersion or suspension described herein, the dispersion or suspension is presented in an ampule, a vial, a prefilled syringe, a pen (single and/or multi-use pen) or an auto-injector.

Another aspect is a process for preparing a sterilized parenteral pharmaceutical dispersion or suspension by sterilizing, e.g., by either aseptic filtration or terminal sterilization, any pharmaceutical dispersion or suspension described herein.

In another embodiment of any pharmaceutical dispersion or suspension described herein, the dispersion or suspension may be shaken for a sufficient time, for example about 3 minutes, about 2 minutes, about 1 minute, about 40 seconds, about 20 seconds or about 10 seconds to resuspend the levothyroxine or its pharmaceutically acceptable salt prior to administration.

Yet another aspect is a process for preparing sterilized levothyroxine (or a pharmaceutically acceptable salt thereof) active pharmaceutical ingredient (API) by:
a) dissolving levothyroxine or its pharmaceutically acceptable salt in a suitable liquid medium,
b) sterilizing the solution obtained in step a) (e.g., by aseptic filtration), and
c) isolating the sterile levothyroxine or its pharmaceutically acceptable salt.

Another embodiment is a process for the preparation of a levothyroxine dispersion or suspension (such as those described herein) comprising aseptically dispersing sterile levothyroxine or its pharmaceutically acceptable salt in a sterile pharmaceutically acceptable carrier, for example water, and optionally one or more pharmaceutically acceptable excipients.

Yet another embodiment is a process for the preparation of a levothyroxine dispersion or suspension comprising aseptically suspending sterile levothyroxine or its pharmaceutically acceptable salt in a sterile pharmaceutically acceptable carrier, for example water or a mixture of water and a non-aqueous liquid, and optionally one or more pharmaceutically acceptable excipients.

Yet another embodiment is a process for the preparation of a levothyroxine dispersion or suspension comprising (a) sterile filtering a pharmaceutically acceptable carrier which optionally further comprises one or more pharmaceutically acceptable excipients to form a carrier solution, and (b) adding the carrier solution to sterile levothyroxine or its pharmaceutically acceptable salt to obtain a sterile dispersion or suspension. The particle size of levothyroxine or its salt in the sterile suspension may optionally be adjusted by various techniques, for example, high pressure homogenization or microfluidization, and optionally additional sterile or sterilized pharmaceutically acceptable excipient or carriers may also be added after the particle size adjustment step.

Yet another embodiment is a process for the preparation of a levothyroxine dispersion or suspension, the process comprising:
a) sterile filtering a pharmaceutically acceptable carrier which optionally further comprises one or more pharmaceutically acceptable excipients to form a sterile carrier solution,
b) sterile filtering levothyroxine or its pharmaceutically acceptable salt dissolved in a pharmaceutically acceptable liquid medium,
c) mixing the sterile carrier solution obtained in step a) with the sterile levothyroxine or its pharmaceutically acceptable salt obtained in step b) to form a mixture,
d) optionally adjusting the particle size of the levothyroxine or its salt in the mixture of step c) (for example by high-pressure homogenizer, high shear, mechanical homogenizer or ultrasonic homogenizer), and
e) optionally adding one or more pharmaceutically acceptable sterile or sterilized excipients or carriers.

Yet another embodiment is a process for the preparation of a sterile levothyroxine dispersion or suspension, the process comprising terminal sterilization of a pharmaceutical dispersion or suspension (such as those described herein).

Yet another embodiment is a process for the preparation of levothyroxine dispersion or suspension comprising dispersing levothyroxine or its salt in an excipient solution, optionally adjusting the pH, followed by adjusting the particle size of the dispersion or suspension by high-pressure homogenizer, high shear, mechanical homogenizer or ultrasonic homogenizer and sterilization of the dispersion or suspension by filtration through a 0.2 or 0.22 micron sterile filter.

Yet another embodiment is a process for the preparation of a levothyroxine dispersion or suspension, the process comprising:
a) dissolving levothyroxine or it's salt in a non-aqueous liquid (such as, e.g., dimethyl sulfoxide (DMSO)) to obtain a solution.
b) Optionally, adding one or more pharmaceutically acceptable excipients (such as, e.g., surfactants, tonicity adjusting agents, buffering agents, and/or stabilizers), to the sterile water for injection (WFI) at 20-25° C. and stirred until dissolution.
c) Adding levothyroxine sodium solution obtained in step a) into the sterile WFI or excipient solution obtained in step b) with stirring to obtain a levothyroxine sodium slurry. Optionally, adding additional pharmaceutically acceptable excipients (such as, e.g., surfactants, tonicity adjusting agents, buffering agents, and/or stabilizers) to the API slurry.
d) Optionally adjusting the pH of the suspension in step c) to the desired target pH using pH adjusting solutions.
e) Optionally reducing the particle size of the suspension obtained in step d) using a high-pressure homogenizer, high shear, mechanical homogenizer or ultrasonic homogenizer
f) Filtering the suspension obtained in step e) through a 0.2 or 0.22 micron sterile filter and
g) Filling the final suspension composition into vials or prefilled syringes, stoppered and sealed. Optionally, the filled vials or prefilled syringes may be lyophilized and/or terminally sterilized.

Yet another embodiment is a method of treating hypothyroidism, myxedema coma and/or another thyroid disorder in a patient in need thereof comprising administering to the patient a pharmaceutical dispersion (such as a pharmaceutical suspension) as described herein. In certain embodiments of any of the methods described herein, the pharmaceutical dispersion is administered once in about 2 days (i.e., once every 2 days), once in about 3 days, once in about 4 days, once in about 5 days, once in about 6 days, once in about a week, once in about a month, once in about 3 months, or once in about 6 months.

### DESCRIPTION OF THE DIAGRAMS

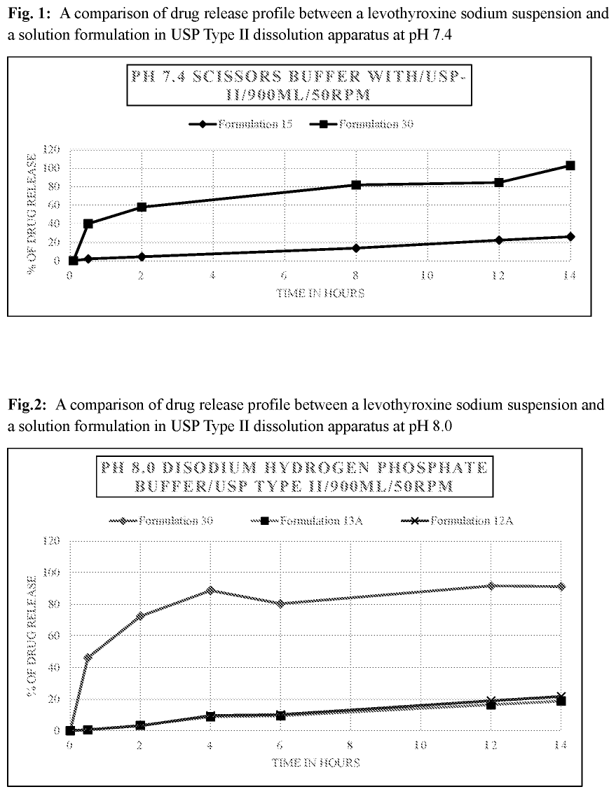

FIG. 1: A comparison of drug release profile between a levothyroxine sodium suspension formulation and a solution formulation in USP Type II dissolution apparatus at pH 7.4.

FIG. 2: A comparison of drug release profile between a levothyroxine sodium suspension formulation and a solution formulation in USP Type II dissolution apparatus at pH 8.0.

### DETAILED DESCRIPTION OF THE INVENTION

In certain embodiments, the pharmaceutical dispersions according to any of the embodiments described herein are long-acting, delayed release, extended release or sustained release compositions. Disperse systems have been broadly classified as systems in which one substance, the dispersed phase, is distributed throughout another substance, the continuous phase or vehicle. Examples of pharmaceutical dosage forms that can be classified as disperse systems are suspensions, emulsions, creams, ointments, pastes, foams, suppositories, and aerosols. In one embodiment, the pharmaceutical dispersion is a suspension for parenteral administration. In one embodiment, the pharmaceutical dispersion is an aqueous suspension. Suspensions generally refer to a dispersed solid phase in a continuous liquid phase. For example, the pharmaceutical dispersion can be a suspension, an emulsion or a lipid nano dispersion.

The pharmaceutical dispersion can provide for the release of the drug (levothyroxine or its pharmaceutically acceptable salt) over an extended period of time. In one embodiment, the release of the drug will occur over a period greater than about 1 day. In another embodiment, the release of the drug will occur over a period greater than about 2 days. In some embodiments, the release of the drug will occur over a period greater than about 3 days. In some embodiments, the release of the drug will occur over a period greater than about 4 days. In some embodiments, the release of the drug will occur over a period greater than about 5 days. In some embodiments, the release of the drug will occur over a period greater than about 6 days. In some embodiments, the release of the drug will occur over a period greater than about 1 week. In some embodiments, the release of the drug will occur over a period greater than about 2 weeks. In some embodiments, the release of the drug will occur over a period greater than about 3 weeks. In some embodiments, the release of the drug will occur over a period greater than about 4 weeks. In some embodiments, the release of the drug will occur over a period greater than about 5 weeks. In some embodiments, the release of the drug will occur over a period greater than about 6 weeks. In some embodiments, the release of the drug will occur over a period greater than about 8 weeks. In some embodiments, the release of the drug will occur over a period greater than about 10 weeks. In some embodiments, the release of the drug will occur over a period greater than about 12 weeks. In some embodiments, the release of the drug will occur over a period greater than about 18 weeks. In some embodiments, the release of the drug will occur over a period greater than about 24 weeks.

The aqueous compositions according to the present invention provide prolonged, sustained or extended release of levothyroxine (or its pharmaceutically acceptable salt) to the general systemic circulation of a patient or to local sites of action in a patient.

In some embodiments, the term “long-acting, delayed release, extended release or sustained release” refers to a release profile wherein the therapeutic effect of levothyroxine or its pharmaceutically acceptable salt, such as levothyroxine sodium, is maintained for an extended period. In some embodiments, the therapeutic effect is maintained for a period greater than about 1 day. In some embodiments, the therapeutic effect is maintained for a period greater than about 2 days. In some embodiments, the therapeutic effect is maintained for a period greater than about 3 days. In some embodiments, the therapeutic effect is maintained for a period greater than about 4 days. In some embodiments, the therapeutic effect is maintained for a period greater than about 5 days. In some embodiments, the therapeutic effect is maintained for a period greater than about 6 days. In some embodiments, the therapeutic effect is maintained for a period greater than about 1 week. In some embodiments, the therapeutic effect is maintained for a period greater than about 2 weeks. In some embodiments, the therapeutic effect is maintained for a period greater than about 3 weeks. In some embodiments, the therapeutic effect is maintained for a period greater than about 4 weeks. In some embodiments, the therapeutic effect is maintained for a period greater than about 5 weeks. In some embodiments, the therapeutic effect is maintained for a period greater than about 6 weeks. In some embodiments, the therapeutic effect is maintained for a period greater than about 8 weeks. In some embodiments, the therapeutic effect is maintained for a period greater than about 10 weeks. In some embodiments, the therapeutic effect is maintained for a period greater than about 12 weeks. In some embodiments, the therapeutic effect is maintained for a period greater than about 18 weeks. In some embodiments, the therapeutic effect is maintained for a period greater than about 24 weeks. In some embodiments, the therapeutic effect is maintained for a period greater than about 30 weeks. In some embodiments, the therapeutic effect is maintained for a period greater than about 1 month. In some embodiments, the therapeutic effect is maintained for a period greater than about 2 months. In some embodiments, the therapeutic effect is maintained for a period greater than about 3 months. In some embodiments, the therapeutic effect is maintained for a period greater than about 4 months. In some embodiments, the therapeutic effect is maintained for a period greater than about 5 months. In some embodiments, the therapeutic effect is maintained for a period greater than about 6 months. In some embodiments, the therapeutic effect is maintained for a period of about 1 day to about 3 months. In some embodiments, the therapeutic effect is maintained for a period of about 1 day to about 6 months. In some embodiments, the therapeutic effect is maintained for a period of about 1 week to about 1 months. In some embodiments, the therapeutic effect is maintained for a period of about 1 week to about 2 months. In some embodiments, the therapeutic effect is maintained for a period of about 1 week to about 3 months. In some embodiments, the therapeutic effect is maintained for a period of about 1 week to about 6 months. In some embodiments, the therapeutic effect is maintained for a period of about 2 weeks to about 3 months. In some embodiments, the therapeutic effect is maintained for a period of about 2 weeks to about 2 months. In some embodiments, the therapeutic effect is maintained for a period of about 2 weeks to about 1 month. In some embodiments, the therapeutic effect is maintained for a period of about 3 weeks to about 3 months. In some embodiments, the therapeutic effect is maintained for a period of about 1 month to about 3 months.

In some embodiments of any of the aqueous compositions described herein, the rate of release of the drug levothyroxine (or its pharmaceutically acceptable salt) from the dosage form may be reduced to maintain therapeutic activity of the drug for a longer period or to reduce any toxic effects associated with a particular dosing of the drug. The aqueous compositions described herein have the advantage of providing patients with a dosing regimen that allows for less frequent dosing, thus enhancing patient compliance. The aqueous compositions described herein may also reduce peak-related side effects associated with the drug and can maintain therapeutic concentrations throughout the dosing period thus avoiding periods of insufficient therapeutic plasma concentrations between doses.

The aqueous compositions according to any of the embodiments described herein provide release of the active ingredient levothyroxine or its pharmaceutically acceptable salt over a prolonged period and therefore they may also be referred to as delayed release, extended release or sustained release compositions. Upon administration, the compositions described herein stay in the body and steadily release levothyroxine or its pharmaceutically acceptable salt, keeping such levels of the active ingredient in the patient's system for a prolonged period, for example about two days to about six months, thereby treating hypothyroidism, myxedema coma and other thyroid disorders during such period.

Levothyroxine as mentioned herein according to various embodiments of the present invention includes levothyroxine and its, hydrates, solvates, anhydrous form thereof, and pharmaceutically acceptable salts of any of the foregoing. Levothyroxine may also be referred as the active pharmaceutical ingredient (API). It preferably includes levothyroxine sodium or its hydrate and solvate forms.

The pharmaceutical dispersion compositions, preferably suspensions, according to any of the embodiments described herein, comprise an aqueous carrier and may be referred to as aqueous dispersion compositions or aqueous suspensions. The aqueous carrier is selected from water or a mixture of water and non-aqueous liquid.

The pharmaceutical dispersions, preferably suspensions, according to any of the embodiments described herein, improve solubility, dissolution rate and bioavailability. They are also used as sustained, extended or delayed release (depot) formulations. Such compositions can be used for long term treatment or long-term prevention (of, e.g., hypothyroidism, myxedema coma and other thyroid disorders), for instance when administered parenterally, e.g., intravenously, intramuscularly or subcutaneously. The dispersion, preferably suspension, compositions can provide effective plasma levels-plasma levels above a minimal therapeutical concentration—for a certain period, such as for at least about 2 days, about 3 days, about 4 days, about 5 days, about 6 days, about 1 week, at least about two weeks, at least about 1 month, at least about two months, at least about three months, at least about four months, at least about five months or at least about six months.

The present invention also provides a parenteral pharmaceutical composition comprising;
a) a therapeutically effective amount of levothyroxine or its pharmaceutically acceptable salts
b) one or more pharmaceutically acceptable excipients, and
c) one or more pharmaceutically acceptable carriers wherein the levothyroxine or its pharmaceutically acceptable salt is primarily dispersed in the pharmaceutically acceptable carrier.

The present invention also provides a composition comprising levothyroxine or its pharmaceutically acceptable salts, such as levothyroxine sodium, wherein the composition is a ready-to-use aqueous dispersion, such as an aqueous suspension. The aqueous suspension may also be obtained by reconstituting a lyophilized powder form of levothyroxine or its pharmaceutically acceptable salts, such as levothyroxine sodium, in a conventional diluent.

In certain embodiments, the aqueous dispersion, such as a suspension, composition has a particle size distribution that is suitable for parenteral administration, preferably IV, SC and IM administration, and acceptable physical and chemical attributes after storage at about 2-8° C. for up to about 6 months, up to about 9 months, up to about 12 months, or up to about 24 months.

In certain embodiments, the aqueous dispersion, such as a suspension, composition has acceptable particle size distribution that is suitable for parenteral administration, preferably IV, SC and IM administration and acceptable physical and chemical attributes after storage at 25° C./60% RH (relative humidity) for up to about 6 months, up to about 9 months, up to about 12 months, or up to about 24 months.

In certain embodiments, the compositions according to the present invention may also be provided in the form of lyophilized powder (freeze dried) for reconstitution for injection. The lyophilized powder has an acceptable assay after storage at about 2-8° C. for up to about 6 months, up to about 9 months, up to about 12 months, or up to about 24 months. The lyophilized powder has an acceptable assay after storage at 25° C./60% RH for up to about 6 months, or up to about 9 months, or up to about 12 months, or up to 24 months.

The lyophilized powder is reconstituted in a diluent such as sterile water for injection, 0.9% sodium chloride, 5% dextrose or lactated Ringer's solution. The reconstitution may be effected by shaking the vial for a few seconds to a few minutes, for example about 10 seconds, about 30 seconds, about 60 seconds, about 2 minutes, about 5 minutes or about 10 minutes. The reconstituted suspension may be administered immediately or stored at refrigerated conditions (e.g., about 2-8° C.) up to about 12 to about 24 hours or stored at room temperature for up to about 12 hours.

The present invention further provides a method of treating hypothyroidism, myxedema coma or other thyroid-related disorders comprising administering to a patient in need thereof a pharmaceutical aqueous dispersion, such as an aqueous suspension, composition comprising a therapeutically effective amount of levothyroxine or its pharmaceutically acceptable salts and one or more pharmaceutically acceptable carriers, such as an aqueous carrier. In the aqueous suspension, the levothyroxine or its pharmaceutically acceptable salt is primarily suspended in the pharmaceutically acceptable carrier.

A “therapeutically effective amount” as used herein is intended to qualify the amount of levothyroxine that will achieve the goal of treating hypothyroidism, myxedema coma or other thyroid-related disorders. The therapeutically effective dose of levothyroxine in the aqueous composition may be equivalent (e.g., bioequivalent) to a daily dose (e.g., oral daily dose) of about 1 mcg to about 3000 mcg, about 10 mcg to about 1000 mcg, about 20 mcg to about 500 mcg, about 25 mcg to about 500 mcg, or about 25 mcg to about 300 mcg. Suitable doses include, but are not limited to, about 10 mcg to about 30 mg for each dosage form. However, it is understood that the amount of levothyroxine administered will be determined by a physician according to various parameters including the route of administration, patient's age and response to the treatment. According to various embodiments, the therapeutically effective amount of levothyroxine is about 50 mcg to about 500 mcg/day which would be considered for weekly, monthly, once in three months or once in six months dosage regimen using IV, SC or IM administration. In certain embodiments, the therapeutically effective amount is about 100 mcg, about 200 mcg, about 300 mcg, about 400 mcg, about 500 mcg, about 600 mcg, about 700 mcg, about 800 mcg, about 900 mcg, about 1000 mcg, about 1100 mcg, about 1200 mcg, about 1300 mcg, about 1400 mcg, about 1500 mcg, about 1600 mcg, about 1700 mcg, about 1800 mcg, about 1900 meg or about 2000 mcg once in 2 days, once in 3 days, once in 4 days, once in 5 days, once in 6 days or once in a week. Alternatively, the therapeutically effective amount of levothyroxine is selected to be equivalent to the effect of once daily oral dosing of about 20 mcg to about 500 mcg.

The term “primarily dispersed” refers to a dispersion in which at least 10% by weight of the levothyroxine or its pharmaceutically acceptable salt is present in particulate or colloid form. In another embodiment, at least 10, 20, 30, 40, 50, 55, 60, 65, 70, 75, 80, 85, 90, or 95% by weight of the levothyroxine or its pharmaceutically acceptable salt (based on 100% by weight of levothyroxine or its pharmaceutically acceptable salt in the dispersion) is present in particulate or colloid form.

The term “primarily suspended” refers to a suspension in which at least 50% by weight of the levothyroxine or its pharmaceutically acceptable salt is suspended (in particulate or colloid form). In another embodiment, at least 55, 60, 65, 70, 75, 80, 85, 90, or 95% by weight of the levothyroxine or its pharmaceutically acceptable salt (based on 100% by weight of levothyroxine or its pharmaceutically acceptable salt in the suspension) is suspended.

Levothyroxine or its pharmaceutically acceptable salts is predominantly suspended in the pharmaceutical carriers. The present invention provides the suspension wherein the amount of levothyroxine or its pharmaceutically acceptable salts dispersed, preferably suspended, in the formulation is about 50%, about 55%, about 60%, about 65%, about 70%, about 75%, about 70%, about 85%, about 90% or about 95% of the total quantity.

Levothyroxine or its pharmaceutically acceptable salts, preferably levothyroxine sodium, can be present in the composition in any suitable concentration. Levothyroxine sodium can be present in the formulation at a concentration between about 20 mcg/mL to about 10 mg/mL, between about 100 mcg/mL and about 7.5 mg/mL, between about 500 mcg/mL to about 5 mg/mL, or between about 1000 mcg/mL to about 3 mg/mL. Typically, levothyroxine sodium can be present in the formulation at a concentration about 25 mcg/ml, about 50 mcg/ml, about 100 mcg/ml, about 150 mcg/ml, about 200 mcg/ml, about 250 mcg/ml, about 300 mcg/ml, about 350 mcg/ml, about 400 mcg/ml, about 450 mcg/ml, about 500 mcg/ml, about 550 mcg/ml, about 600 mcg/ml, about 650 mcg/ml, about 700 mcg/ml, about 750 mcg/ml, about 800 mcg/ml, about 850 mcg/ml, about 900 mcg/ml, about 950 mcg/ml, about 1000 mcg/ml, about 1100 mcg/ml, about 1200 mcg/ml, about 1300 mcg/ml, about 1400 mcg/ml, about 1500 mcg/ml, about 1600 mcg/ml, about 1700 mcg/ml, about 1800 mcg/ml, about 1900 mcg/ml or about 2000 mcg/ml.

The median particle size of the dispersed levothyroxine or its pharmaceutically acceptable salt is suitable for parenteral administration, preferably IV, SC and IM administration and may be selected based on the intended duration of therapy. For example, in one embodiment, it is in the range of about 50 nm to about 75 μm. In some embodiments, the median particle size is about 100 nm to about 75 μm, such as from about 20 or 40 μm to about 75 μm, from about 100 nm to about 2500 nm, from about 100 to about 500 nm, or about 1500 to about 2500 nm. In some embodiments, the median particle size is about 100 nm to about 50 μm. In another embodiment, the median particle size is about 300 nm to about 25 μm. In another embodiment, the median particle size is about 500 nm to about 15 μm. In another embodiment, the median particle size is about 1 μm to about 10 μm. In another embodiment, the median particle size is about 1.5 to about 5 μm. In another embodiment, the median particle size is about 2 μm to about 3 μm. In another embodiment, the median particle size is less than about 3000 nm, less than about 2750 nm, less than about 2500 nm, less than about 2250 nm, less than about 2000 nm, less than about 1750 nm, less than about 1500 nm, less than about 1250 nm, less than about 1000 nm, less than about 900 nm, less than about 750 nm, less than about 500 nm, less than about 250 nm, less than about 100 nm, less than about 50 nm or less than about 25 nm.

As used herein, the term median particle size has its conventional meaning as known to the person skilled in the art and can be measured by art-known particle size measuring techniques such as, for example, sedimentation field flow fractionation, photon correlation spectroscopy, laser diffraction or disk centrifugation. The median particle sizes mentioned herein may be related to volume distributions of the particles. In that instance, by “a median particle size of less than about 100 nm” it is meant that at least 50% of the volume of the particles has a particle size of less than about 100 nm, and the same applies to the other particle sizes mentioned. In a similar manner, the median particle sizes may be related to weight distributions of the particles. In that instance, by “a median particle size of less than about 100 nm” it is meant that at least 50% of the weight of the particles has a particle size of less than about 100 nm, and the same applies to the other particle sizes mentioned. In general, volume and weight distribution result in the same or about the same value for the median particle size. Unless otherwise specified, the median particle size is based on a volume distribution as measured by laser diffraction or dynamic light scattering.

The particle size of levothyroxine or its salt in the dispersion, such as a sterile suspension, may optionally be adjusted by various techniques, for example high pressure homogenization or microfluidization, and optionally additional sterile or sterilized pharmaceutically acceptable excipients and/or carriers may also be added after the particle size adjustment step.

In additional embodiments, the pharmaceutically acceptable salt of levothyroxine is selected from levothyroxine sodium and levothyroxine potassium.

The aqueous suspension compositions according to any of the embodiments described herein may further comprise one or more of a suspending agent, a surfactant, a pH adjusting agent, a buffering agent, a stabilizing agent, a preservative, an anti-oxidant, a chelating agent, a tonicity agent, a viscosity enhancing agent, a release retardant, or any combination of any of the foregoing. Certain ingredients may function as two or more of these agents simultaneously, e.g., behave like a preservative and a buffer, or behave like a buffer and an isotonizing agent.

In certain embodiments, the suspending agent is selected from cellulose derivatives, e.g., methyl cellulose, hydroxyethyl cellulose, hydroxypropyl methyl cellulose and carboxymethyl cellulose or its sodium salt, polyvinylpyrrolidone (povidone), alginates, chitosan, dextrans, gelatin, polyethylene glycols, polyoxyethylene- and polyoxy-propylene ethers, and any combination of any of the foregoing. In certain embodiments, the suspending agent may also act as a viscosity enhancer.

The surfactant is selected from ionic, non-ionic or amphoteric surfactants. In certain embodiments, the surfactant is selected from polyoxyethylene derivatives of sorbitan esters, polyoxyethylene- and polyoxypropylene ethers, ethers of fatty alcohols with polyoxyalkylene glycols, free fatty acids, esters of fatty acids with polyoxyalkylene compounds like polyoxypropylene glycol and polyoxyethylene glycol; soaps; glycerol-polyalkylene stearate; glycerol-polyoxyethylene ricinoleate; homo- and copolymers of polyalkylene glycols; polyethoxylated soya-oil and castor oil as well as hydrogenated derivatives; ethers and esters of sucrose or other carbohydrates with fatty acids, fatty alcohols, these being optionally polyoxyalkylated; mono-, di- and triglycerides of saturated or unsaturated fatty acids; glycerides or soya-oil and sucrose; sodium caprolate, ammonium sulfate, sodium dodecyl sulfate (SDS), Triton-100 and anionic surfactants containing alkyl, aryl or heterocyclic structures and any combination of any of the foregoing. Preferred surfactants include but not limited to polysorbate 20, polysorbate 80, lecithin, sodium deoxycholate, tyloxapol, pluronics, poloxamers, polyoxyethylene 20 cetyl ether, cremophor EL, cetyl trimethylammonium bromide (CTAB) and anionic surfactants containing alkyl, aryl or heterocyclic structures, and cyclodextrins.

The pharmaceutical compositions of the present invention may also contain pH adjusting agents or neutralizing agents. The pH adjusting agent or neutralizing agent may be selected from sodium hydroxide, potassium hydroxide, magnesium hydroxide, sodium carbonate, Tris (tris(hydroxymethyl)aminomethane), sodium linoleate, sodium oleate, potassium carbonate, potassium linoleate, potassium oleate, triethylamine, tromethamine, hydrochloric acid, sulfuric acid, nitric acid, acetic acid, tartaric acid, citric acid, lactic acid or any combination of any of the foregoing.

The pharmaceutical compositions may contain a buffering agent, which is used to resist change in pH upon dilution or addition of acid or alkali. The buffering agent is suitable to maintain the pH from 3 to 11. The buffering agent may be selected from, by way of example and without limitation, formic acid, acetic acid, sodium acetate, citric acid, sodium citrate, adipic acid, benzoic acid, sodium benzoate, maleic acid, monobasic sodium phosphate, dibasic sodium phosphate, disodium hydrogen phosphate dodecahydrate, lactic acid, tartaric acid, succinic acid, potassium metaphosphate, potassium phosphate, monobasic sodium acetate, sodium bicarbonate, sodium tartrate, sodium succinate, malate, formate, pyridine, TRIS, HEPES (4-(2-hydroxyethyl)-1-piperazineethanesulfonic acid), biological buffers, amino acids such as arginine, alanine, histidine, glycine and lysine; meglumine, borate or any combination of any of the foregoing. Hydrate and anhydrate forms of the buffering agents also can be used in the present invention.

In certain embodiments, the viscosity enhancing agent is selected from cellulose derivate selected from hydroxy ethyl cellulose, carboxy cellulose derivative or it is salt, xanthan gum, guar gum and the like. In some embodiments, the viscosity enhancing agent may also act as a suspending agent.

In certain embodiments, the chelating agent is selected from acetic acid, citric acid, ethylenediaminetetraacetic acid (EDTA) or its sodium salt and the like, and combinations thereof.

The pharmaceutical compositions may contain a stabilizing agent selected from, for example, sodium iodide, potassium iodide, tromethamine, ethylenediaminetetraacetic acid (EDTA) or its sodium salt, amino acids such as glycine, lysine, aspartic acid, methionine or their salts and the like, TTTA (1,7,13-triaza-4,10,16-trioxacyclooctadecane-N,N′,N″-triacetate), DOTRP (tetraethyleneglycol-1,5,9-triazacyclododecane-N,N′,N″-tris(methylene phosphonic acid), and EGTA (ethylene glycol-bis(β-aminoethyl ether)-tetraacetic acid), DOTA (1,4,7,10-tetraazacyclododecane-1,4,7,10-tetraacetic acid), tetrasodium-iminodisuccinate (IDS), tartaric acid, citric acid, acetic acid, ascorbic acid, formic acid, succinic acid, pentetic acid, tannic acid, diethylenetriamine pentaacetate (DTPA) or its salts, trisodium ethylenediamine disuccinate and the like, and combinations thereof. The stabilizing agents also may function as a chelating agent.

The pharmaceutical compositions may contain a “tonicity modifier” that can be used to adjust the tonicity of the liquid formulation. Suitable tonicity modifiers include, for example, glycerine, lactose, mannitol, dextrose, sodium chloride, sodium sulphate, sorbitol, trehalose, propylene glycol and others known to those of ordinary skill in the art, and any combination thereof. In one embodiment, the tonicity of the liquid formulation approximates that of the tonicity of blood or plasma. The amount of tonicity modifier may range from about 1 mg/mL to about 50 mg/ml of the composition, such as from about 5-10 mg/mL of the composition. In certain embodiments, the composition may contain sodium chloride at a concentration of about 5 mg/mL to about 15 mg/mL, such as a concentration of about 5 mg/mL to about 10 mg/mL, or a concentration of about 9 mg/mL. In certain embodiments, the composition will have an osmolality between about 200 to about 600 mOsm/kg, or between 200 to about 400 mOsmol/kg, or between about 270 to about 340 mOsm/kg.

The compositions described herein may optionally comprise one or more preservatives. The term “preservative” refers to a substance present in a composition which can, at least in part, prevent and/or reduce the decomposition of the composition. In some embodiments, the preservative may prevent and/or reduce the decomposition of the composition by microbial growth in the composition. Preferably, the preservative is pharmaceutically acceptable.

Non limiting examples of preservatives include one or more of benzalkonium chloride, benzealkonium chloride, benzoic acid, benzyl alcohol, benzyl paraben, bronopol, butyl paraben, cetrimide, cetylpyridinium chloride, chlorbutanol, chlorhexidine, chlorocresol, chloroxylenol, cresol, ethyl alcohol, ethyl paraben, ethylparaben, glycerin, hexetidine, imidurea, isobutyl paraben, meta-cresol, methyl paraben, methylparaben, phenol, thimerosal, and any combination thereof.

In certain embodiments, the antioxidant is provided in a stabilizing amount. In certain non-limiting embodiments, the antioxidant or stabilizing agent may include methionine, glycerol, monothioglycerol, sodium bisulfite, sodium metabisulfite, propylene glycol, phenol, EDTA, butylated hydroxy toluene (BHT), butylated hydroxy anisole (BHA), tocopherol, and any combination thereof. The term “methionine” as used herein encompasses both L-methionine and D-methionine. Examples of antioxidants and stabilizing agents may also include, without limitation, acetone, tocopherol, sodium bisulfate, ascorbic acid, ascorbyl palmitate, citric acid, glycine, L-cysteine hydrochloride, D-methionine, L-methionine, butylated hydroxy anisole, butylated hydroxytoluene, hydro phosphorous acid, monothioglycerol, propyl gallate, sodium ascorbate, sodium citrate anhydrous, sodium citrate dihydrate, sodium sulfide, sodium sulfite, sodium bisulfite, sodium formaldehyde sulfoxylate, thioglycolic acid, sodium metabisulfite and others known to those of ordinary skill in the art, and any combination thereof.

In certain embodiments, the pharmaceutically acceptable carrier comprises water or a mixture of water and non-aqueous liquid. Suitable non-aqueous liquids include protic and aprotic solvents, but are not limited to, ethanol, tertiary butyl alcohol, acetonitrile, acetone, propylene glycol (PG), polyethylene glycol (PEG), glycerin, N-methyl formamide, N,N-dimethyl formamide (DMF), N,N-dimethylacetamide (DMA), dimethyl sulfoxide (DMSO), N-methyl pyrrolidone (NMP), 2-pyrrolidone and any combination of any of the foregoing. Preferably, the carrier is selected from a mixture of water and one or more non-aqueous liquids, for example, a mixture of water and DMSO, a mixture of water, DMSO and PEG, or a mixture of water, DMSO and PG. In a preferred embodiment, the water is water for injection.

In another embodiment, the carrier is selected from a non-aqueous liquid such as ethanol, propylene glycol (PG), polyethylene glycol (PEG), glycerin, acetone, N-methyl formamide, N,N-dimethylformamide (DMF), N,N-dimethylacetamide (DMA), dimethyl sulfoxide (DMSO), N-methyl pyrrolidone (NMP), 2-pyrrolidone, or any combination of any of the foregoing. In one embodiment, the carrier which is a non-aqueous liquid does not contain water.

In another embodiment, the present invention provides an aqueous suspension comprising a therapeutically effective amount of levothyroxine or its pharmaceutically acceptable salt, a pharmaceutically acceptable carrier comprising water and one or more pharmaceutically acceptable excipients, wherein the levothyroxine is primarily suspended in the carrier. In another embodiment, the pharmaceutical carrier is a mixture of water and DMSO.

In another embodiment, the present invention provides an aqueous suspension comprising levothyroxine sodium, a pharmaceutically acceptable carrier comprising a mixture of DMSO, PEG and water and one or more pharmaceutically acceptable excipients, wherein the levothyroxine sodium is primarily suspended in the carrier.

In another embodiment, the present invention provides a method of treating hypothyroidism myxedema coma or other thyroid disorders by administering the aqueous suspension according to any of the embodiments described herein, wherein the method may involve an initiation dose and one or more maintenance doses. The initiation dose may involve administration of a currently available oral or injection dose for about 7 days, about 3 days or about 2 days, or administration of the long-acting compositions described herein. The maintenance dose involves an administration of an aqueous suspension according to the present invention containing a therapeutically effective amount of levothyroxine or its pharmaceutically acceptable salts.

The present invention further provides a process for the preparation of a pharmaceutical composition, the process comprising first preparing a sterile levothyroxine or its pharmaceutically acceptable salt thereof by:
a) dissolving levothyroxine or its pharmaceutically acceptable salt in a suitable liquid medium,
b) sterilizing the solution obtained in step a), and
c) isolating the sterile levothyroxine or its pharmaceutically acceptable salt.

In certain embodiments, the pharmaceutically acceptable salt is selected from sodium, potassium etc.

In certain embodiments, the liquid medium used in step a) is selected from water or a mixture of water and an organic solvent. The liquid medium may be selected from an alcohol such as ethanol, tertiary butyl alcohol, propylene glycol (PG), polyethylene glycol (PEG), glycerin, acetone, N-methylformamide, N,N-dimethylformamide (DMF), N,N-dimethylacetamide (DMA), dimethylsulfoxide (DMSO), N-methylpyrrolidone (NMP), 2-pyrrolidone and the like or any combination thereof.

In certain embodiments, the solution obtained in step b) is filtered in an aseptic environment. For example, the solution may be filtered through a 0.2 or 0.22 micron filter to obtain the sterile API.

Isolation of levothyroxine or its pharmaceutically acceptable salt may be performed by conventional methods such as precipitation or a solvent evaporation method using a rotavapor.

Precipitation may involve cooling the filtrate or addition of an anti-solvent to the filtrate. The solid obtained from the above process may be subjected to micronization by various techniques, for example high pressure homogenization or microfluidization to obtain the desired median particle size.

In another embodiment, the present invention provides a process for preparation of pharmaceutical dispersion composition comprising aseptically suspending sterile levothyroxine or its pharmaceutically acceptable salt in a sterile pharmaceutically acceptable carrier, for example water or a mixture of water and a non-aqueous liquid, and optionally containing pharmaceutically acceptable excipients. In certain embodiments, the ratio of water and the non-aqueous liquid is in the range of about 0.1:99.9 to about 99.9:0.1.

In another embodiment, the present invention provides a process for the preparation of a pharmaceutical dispersion composition comprising sterile filtering a pharmaceutically acceptable carrier which optionally contains one or more pharmaceutically acceptable excipients and addition of the sterile carrier/excipient solution to sterile levothyroxine or its pharmaceutically acceptable salt to obtain a sterile suspension. The particle size of levothyroxine or its salt in the sterile suspension may optionally be adjusted by various techniques, for example high pressure homogenization or microfluidization, and optionally additional sterile or sterilized pharmaceutically acceptable excipient or carriers may also be added after the particle size adjustment step.

In another embodiment, the present invention provides a process for the preparation of a pharmaceutical composition, the process comprising
a) sterile filtration of a pharmaceutically acceptable carrier which optionally contains one or more pharmaceutically acceptable excipients,
b) sterile filtration of levothyroxine or its pharmaceutically acceptable salt dissolved in a pharmaceutically acceptable liquid medium,
c) mixing the sterile carrier/excipient solution obtained in step a) to the sterile levothyroxine or its pharmaceutically acceptable salt obtained in step b), resulting in the precipitation of the levothyroxine (or its pharmaceutically acceptable salt).
d) optionally adjusting the particle size of the levothyroxine or its pharmaceutically acceptable salt in the formulation by various techniques, for example high-pressure homogenizer, high shear, mechanical homogenizer or ultrasonic homogenizer,
e) optionally adding one or more pharmaceutically acceptable sterile or sterilized excipients or carriers,
f) filling into a pharmaceutically acceptable container closure and/or delivery system, such as vials, prefilled syringes, cartridges, and/or autoinjectors,
g) optionally lyophilizing the filled drug product, and
h) optionally terminally sterilizing the drug product in its final container (either with or without the optional lyophilization step)

In certain embodiments, the carrier used in step a) is selected from water or a mixture of water and a non-aqueous liquid. The non-aqueous liquid may be selected from protic and aprotic solvents, but not limited to an alcohol such as ethanol, tertiary butyl alcohol, acetonitrile, acetone, propylene glycol (PG), polyethylene glycol (PEG), glycerin, N-methylformamide, N,N-dimethyl formamide (DMF), N,N-dimethylacetamide (DMA), dimethylsulfoxide (DMSO), N-methylpyrrolidone (NMP), 2-pyrrolidone and the like or any combination thereof. The pharmaceutically acceptable liquid medium may include one or more of the same components as the pharmaceutically acceptable carrier.

In another embodiment, the present invention provides a process for the preparation of a sterile pharmaceutical composition, the process comprising terminal sterilization of an aqueous suspension comprising a therapeutically effective amount of levothyroxine or its pharmaceutically acceptable salt in a pharmaceutically acceptable carrier and one or more pharmaceutically acceptable excipients, wherein the levothyroxine or its pharmaceutically acceptable salt is primarily suspended in the pharmaceutically acceptable carrier, such as an aqueous carrier.

The term “sterile suspension” as used herein relates to a suspension used for parenteral administration which is free from all viable microbial contamination.

In certain embodiments, the levothyroxine or its pharmaceutically acceptable salt is present in the sterile composition in an amount within the range from about 0.01% to about 50% (w/v), based on the total composition.

The sterile levothyroxine compositions described herein may be prepared using sterile filtration and aseptic processing. Alternately, the levothyroxine composition may be subjected to bulk sterilization by moist heat prior to filling into a container. The levothyroxine compositions described herein may also be terminally sterilized in the final container using moist heat or dry heat or gamma or e-beam sterilization. In one embodiment, moist heat sterilization may be performed by using sterilization in place (SIP) system. This step may also be referred to as steam sterilization.

In certain embodiments, a dispersion, such as a sterile suspension, obtained by the processes described herein further undergoes homogenization and milling to afford the desired particle size.

The term “homogenization” as used herein refers to the process used to prepare a uniform sterile aqueous suspension. Homogenization may be performed by using either mechanical or ultrasonic processes. In certain embodiments, the aqueous suspension is optionally homogenized using a microfluidizer or a high-pressure homogenizer at optimized pressure and temperature.

The term “milling” as used herein refers to the process which is used to prepare uniform particle size of sterile suspension. Milling may be performed using high-pressure homogenizer, high shear, mechanical homogenizer or ultrasonic homogenizer. Specifically, the milling may be performed using bead mill, ball mill, roller mill, Netzsch mill, DC mill or planetary mill. The milling procedure and equipment employed are used to afford the desired mean particle size of levothyroxine or its pharmaceutically acceptable salt particles in the sterile suspension or composition.

In certain embodiments, a sterilized and optionally homogenized suspension formulation as described herein may be lyophilized to obtain a lyophilized cake or powder, which is easy to reconstitute to form a uniform and homogeneous suspension. Alternately, a terminal sterilization step such as gamma sterilization or e-beam sterilization may be carried out after lyophilization. Conventional diluents such as water, 0.9% sodium chloride, 5% dextrose, etc., may be used for reconstitution of the lyophilized powder to obtain a suspension prior to administration.

In another embodiment, the pharmaceutical suspensions according to the present invention comprise levothyroxine or its pharmaceutically acceptable salt in a liquid solution form that is mixed with a pharmaceutically acceptable carrier to form a composition, wherein the levothyroxine or its pharmaceutically acceptable salt is primarily suspended in the pharmaceutically acceptable carrier. The solution of levothyroxine or its pharmaceutically acceptable salt may be provided in a vial, a dual chamber syringe or vial or cartridge, wherein the solution (solution of levothyroxine or its pharmaceutically acceptable salt in one chamber and the pharmaceutically acceptable carrier in a second chamber). The solution and carrier for preparation of the final composition may be supplied in separate vials. The present invention also provides a kit containing the solution of levothyroxine or its pharmaceutically acceptable salt in a vial and the pharmaceutically acceptable carrier in a vial or a prefilled syringe (PFS)

In certain embodiments, the pharmaceutical compositions according to the present invention wherein levothyroxine or its pharmaceutically acceptable salt is dispersed, particularly primarily suspended, in the pharmaceutically acceptable carrier may be presented in an ampule, vial, prefilled syringe, pen (single and multi-use pen) and/or auto-injector.

In another aspect, a pharmaceutical dispersion composition suitable for parenteral administration is provided which comprises a stable emulsion comprising an oil phase, wherein the oil phase comprises levothyroxine or its pharmaceutically acceptable salt, a surfactant and a co-surfactant; and an aqueous phase, wherein the aqueous phase comprises water, a tonicity agent and a pH-adjusting agent. The composition is an oil-in-water emulsion comprising an oil selected from the group consisting of structurally modified or hydrolyzed coconut oil, olive oil, soybean oil, safflower oil, triglycerides, octyl and decyl glycerate, ethyl oleate, glyceryl linoleate, ethyl linoleate, glyceryl oleate, cholesteryl oleate/linoleate or any mixture thereof. The emulsion comprises an emulsifier, a co-surfactant, a tonicity agent, a pH adjusting agent, a buffering agent and water.

In additional embodiments, any of the compositions described herein contains less than about 5% w/w of liothyronine, such as less than about 4% w/w of liothyronine, less than about 3% w/w of liothyronine, less than about 2% w/w of liothyronine, less than about 1% w/w of liothyronine, less than about 0.5% w/w of liothyronine, less than about 0.1% w/w of liothyronine, when stored, for example, at 40±2° C./75±5% RH or 25±2° C./60±5% RH for 3 months or 6 months.

In additional embodiments, any of the compositions described herein contains less than about 5% w/w of total impurities, such as less than about 4% w/w of total impurities, less than about 3% w/w of total impurities, less than about 2% w/w of total impurities, or less than about 1% w/w of total impurities, when stored, for example, at 40±2° C./75±5% RH or 25±2° C./60±5% RH for 3 months or 6 months.

Some embodiments of the present disclosure will be described in greater detail by way of specific examples. The following examples are offered for illustrative purposes and are not intended to limit the present invention in any manner.

### EXAMPLES

### Example 1

### Generalized Procedure for the Preparation of Sterile Levothyroxine Sodium Aqueous Suspensions by Precipitation

Sterile aqueous suspensions of levothyroxine sodium were prepared as per the following general procedure:
a) Levothyroxine sodium was first dissolved in a non-aqueous liquid (example dimethyl sulfoxide (DMSO)) to obtain a solution (example 100 to 500 mg/mL). The solution was sterile filtered through a 0.2 or 0.22 micron filter.
b) Optionally, one or more pharmaceutically acceptable excipients (such as surfactants, tonicity adjusting agents, buffering agents, and/or stabilizers), were added to sterile water for injection (WFI) at 20-25° C. and stirred until dissolution. Further, the pH of the excipient solution may be adjusted to a desired target pH using pH adjusting solutions. The WFI or the excipient solution was sterile filtered through a 0.2 or 0.22 micron filter.
c) The sterile levothyroxine sodium solution obtained in step a) was added into the sterile WFI or excipient solution obtained in step b) with stirring to obtain a levothyroxine sodium suspension.
d) Optionally, the pH of the suspension was adjusted to a desired target pH using pH adjusting agents.
e) The final volume was made up using sterile WFI or excipient solution, and the suspension was filled into a vial and stoppered.
f) Optionally, the filled vials are lyophilized and/or terminally sterilized.

Table 1 summarizes different exemplary formulations of aqueous levothyroxine sodium suspensions prepared by the generalized procedure of Example 1 detailed above. For all formulations disclosed in Table 1, a white to off-white aqueous suspension of levothyroxine sodium was observed.

TABLE 1
Sterile Levothyroxine Sodium Aqueous Suspension Formulations Prepared by Precipitation

| Formulation # | 1a-1f | 2a-2e | 3 | 4 | 6 | 7 | 8 | 9 | 10 | 11 |
|---|---|---|---|---|---|---|---|---|---|---|
| Levothyroxine | 0.5-10 | 0.5-5 | 1 | 3.3 | 3.3 | 1 | 1 | 1 | 1 | 1 |
| sodium (mg) | | | | | | | | | | |
| Dimethyl | 0.5-10 | 0.5-5 | 1 | 6.6 | 6.7 | 10 | 10 | 10 | 10 | 5 |
| sulfoxide (mg) | | | | | | | | | | |
| Polysorbate | — | 2 | 2 | 6.6 | 6.7 | 2 | 2 | 2 | 3 | 0.5 |
| 80 (mg) | | | | | | | | | | |
| Sodium | — | — | 3 | — | — | 3 | 3 | 3 | 3 | 3 |
| chloride (mg) | | | | | | | | | | |
| Mono basic | — | — | 0.5 | — | — | 0.5 | 0.5 | 0.5 | 0.7 | — |
| sodium | | | | | | | | | | |
| phosphate | | | | | | | | | | |
| monohydrate (mg) | | | | | | | | | | |
| Sodium | — | — | 0.4 | — | — | 0.4 | 0.4 | 0.4 | 1.1 | — |
| dihydrogen | | | | | | | | | | |
| phosphate | | | | | | | | | | |
| dihydrate (mg) | | | | | | | | | | |
| Citric acid | — | — | — | — | — | — | — | — | — | 8.7 |
| monohydrate (mg) | | | | | | | | | | |
| Trisodium | — | — | — | — | — | 1 | 1 | 1 | — | 17 |
| Citrate (mg) | | | | | | | | | | |
| Methionine (mg) | — | — | — | — | — | 1 | 1 | 1 | — | — |
| Ethylenediaminetetraacetic | — | — | — | — | — | — | — | — | 0.5 | 0.2 |
| acid (mg) | | | | | | | | | | |
| Polyethylene | — | — | 18 | — | — | — | — | — | — | — |
| glycol 400 (mg) | | | | | | | | | | |
| Sodium | — | — | — | — | — | — | — | — | — | 5 |
| carboxymethyl | | | | | | | | | | |
| cellulose (mg) | | | | | | | | | | |
| Propylene | — | — | — | — | — | 15 | 15 | 15 | — | — |
| glycol (mg) | | | | | | | | | | |
| Hydrochloric | — | — | — | Qs to | — | — | — | — | — | — |
| Acid | | | | pH 7 | | | | | | |
| Sodium | — | — | — | — | — | — | QS to | QS to | — | — |
| Hydroxide | | | | | | | pH 7 | pH 8 | | |
| Water | Qs to | Qs to | Qs to | Qs to | Qs to | Qs to | Qs to | Qs to | Qs to | Qs to |
| | 1 mL | 1 mL | 1 mL | 1 mL | 1 mL | 1 mL | 1 mL | 1 mL | 1 mL | 1 mL |
| Appearance | WS | WS | WS | WS | WS | WS | WS | WS | WS | WS |
| after QS | | | | | | | | | | |
| Optional | Sonication | Sonication | — | — | Sonication | — | — | — | — | — |
| Additional | | | | | | | | | | |
| Dispersion | | | | | | | | | | |
| Process | | | | | | | | | | |
| pH | — | — | 6.0 | 7.0 | 9.6 | 6.5 | 7.2 | 8.4 | — | 6.1 |
| Appearance | HS | HS | — | — | WS | — | — | — | — | WS |
| after dispersion | | | | | | | | | | |

WS: White Suspension;
HS: Hazy Suspension;
QS: Quantity Sufficient

One exemplary aqueous suspension formulation prepared as per the generalized procedure of Example 1 (Formulation 11) was filled in a 2 mL USP Type I glass vial from Schott Kaisha, stoppered using 13 mm Omniflex stopper FM457/0 from Datwyler, sealed and also placed on stability under ICH stability conditions, including accelerated stability (40±2° C./75±5% RH) and long-term stability (25±2° C./60±5% RH). The following attributes were tested: Description, Assay (of levothyroxine sodium), Impurities, and Particle Size. Table 2 summarizes the stability data which show that the formulation is chemically stable after 1 months at 40° C. and after 6 months at 25° C. Particularly surprising is that the levels of liothyronine, the primary degradant in the liquid state, is very low even in the absence of sodium iodide often used to stabilize solution formulations of levothyroxine from deiodination.

TABLE 2
Stability Data for Formulation 11 of Example 1

| | Stability Condition | | | | | |
|---|---|---|---|---|---|---|
| | Initial | 25° C/60% RH | | | 40° C/75% RH | |
| Stability Duration | N/A | 1 M | 3 M | 6 M | 1 M | 3 M |
| Description | WS | WS | WS | WS | WS | WS |
| Median Particle | 60.2 | ND | 57.4 | 57.1 | ND | 53.2 |
| Size (μm) | | | | | | |
| Assay (mg/mL) | 1.05 | 1.01 | 1.01 | 0.903 | 1.06 | 0.75 |
| Liothyronine (% w/w) | 0.02 | 0.02 | 0.05 | 0.09 | 0.09 | 0.11 |
| Total Impurities (% w/w) | 0.36 | 0.55 | 0.81 | 1.03 | 0.92 | 2.59 |

N/A = Not Applicable:
ND = Not Determined;
M = Month(s);
WS = White Suspension

### Example 2

### Generalized Procedure for the Preparation of Levothyroxine Sodium Suspensions by Direct Dispersion

Aqueous suspensions of levothyroxine sodium were prepared by direct dispersion of levothyroxine sodium in a pharmaceutically acceptable carrier with optional pharmaceutical excipients as per the following general procedure:
a) Optionally, one or more pharmaceutically acceptable excipients (such as, e.g., surfactants, tonicity adjusting agents, buffering agents, and/or stabilizers), were added to sterile water for injection (WFI) at 20-25° C. and stirred until dissolution. Further, the pH of the excipient solution may be adjusted to a desired target pH using pH adjusting solutions. The WFI or the excipient solution was sterile filtered through a 0.2 or 0.22 micron filter.
b) Levothyroxine sodium API was added to the WFI or excipient solution obtained in step a) to obtain a slurry. The concentration of the levothyroxine sodium API in the slurry was higher than the intended final concentration of the composition (for example 2 mg/mL to 50 mg/mL). For preparation of sterile suspensions, sterile levothyroxine sodium API can be used.
c) The API slurry obtained in step b) was subjected to particle size reduction by using a high-pressure homogenizer, high shear, mechanical homogenizer or ultrasonic homogenizer
d) Optionally, a separate sterile excipient solution with any additional pharmaceutically acceptable excipients (such as, e.g., surfactants, tonicity adjusting agents, buffering agents, stabilizers, suspending agents, viscosity enhancing agents and/or pH adjustors) was prepared by dissolving them in sterile WFI and sterile filtering through a 0.2 or 0.22 micron filter.
e) The sterile API suspension obtained in step c) was added to the sterile excipient solution obtained in step d).
f) Optionally, the pH of the suspension was adjusted to the desired target pH using pH adjusting solutions.
g) The final volume was made up with sterile WFI or sterile excipient solution and the final suspension composition was filled into vials, stoppered and sealed.
h) Optionally, the filled vials may be lyophilized and/or terminally sterilized.

Table 3 summarizes different exemplary formulations of aqueous levothyroxine sodium suspensions prepared by the generalized procedure as outlined previously in Example 2. For the formulations disclosed in Table 3, a white to off-white aqueous suspension of levothyroxine sodium was observed.

TABLE 3
Sterile Levothyroxine Sodium Aqueous Suspension
Formulations Prepared by Direct Dispersion Using
GEA PANDA PLUS 2000 High Pressure Homogenizer

| Formulation # | 12A | 12B |
|---|---|---|
| Levothyroxine sodium (mg) | 1 | 1.0 |
| Polysorbate 80 (mg) | 0.5 | 0.5 |
| Citric acid monohydrate (mg) | 8.7 | 8.7 |
| Tri sodium citrate dihydrate (mg) | 17.0 | 17.0 |
| Sodium chloride (mg) | 3.0 | 3.0 |
| Ethylenediaminetetraacetic acid (mg) | 0.2 | 0.2 |
| Sodium carboxymethyl cellulose (mg) | 5.0 | — |
| Povidone (mg) | — | 5.0 |
| Water | Qs to 1 mL | Qs to 1 mL |
| NaOH | — | Qs to pH |
| pH | 4.7 | 4.6 |
| Osmolality (mOsmol/kg) | 310 | 315 |

Attributes

| | White | White |
|---|---|---|
| Description | Suspension | Suspension |
| Viscosity (cP) | 2.9 | 1.1 |
| Median Particle Size (nm) | 266 | 2330 |
| Assay (mg/mL) | 0.89 | 0.90 |
| Liothyronine (% w/w) | 0.01 | 0.01 |
| Total Impurities (% w/w) | 0.35 | 0.36 |

QS: Quantity Sufficient

The exemplary aqueous suspension formulations 12A and 12B, prepared as per the generalized procedure outlined previously in Example 2 were, filled in a 2 mL USP Type I glass vial from Schott Kaisha, stoppered using 13 mm Omniflex stopper FM457/0 from Datwyler, sealed, and placed on stability at 60° C. as well as under ICH accelerated (40±2° C./75±5% RH) and long-term (25±3° C./60±5% RH) stability conditions. The following attributes were tested: Description, Viscosity, Assay (of levothyroxine sodium), Impurities, and Particle Size. Table 4 summarizes the stability data for these two formulations (12A and 12B). The data shows that the formulation 12A containing sodium carboxy methylcellulose (sodium CMC) and formulation 12B containing povidone is physically and chemically stable after 1 month at 60° C. and after 3 for 6 months at 40° C. and 25° C. The median particle size of the formulations at 6 months time point for both 25° C. and 40° C. conditions is essentially similar to the initial time point. Also, assay values and corresponding degradation remains essentially similar for both 25° C. and 40° C. conditions.

Particularly surprising again for both the formulations is that the levels of liothyronine, the primary degradant in the liquid state, is very low even in the absence of sodium iodide often used to stabilize solution formulations of levothyroxine from de-iodination.

TABLE 4
Consolidated Stability Data for Formulations of Example 2

| | Stability Condition | | | | | | | | | | | |
|---|---|---|---|---|---|---|---|---|---|---|---|---|
| | Initial | 60° C. | | | 25° C/60% RH | | | | 40° C/75% RH | | | |
| | Stability Duration | | | | | | | | | | | |
| Attributes | N/A | 2 Days | 15 Days | 1 M | 1 M | 2 M | 3 M | 6 M | 1 M | 2 M | 3 M | 6 M |
| | Formulation 12A | | | | | | | | | | | |
| Description | WS | WS | WS | WS | WS | WS | WS | WS | WS | WS | WS | WS |
| Viscosity (cps) | 2.9 | ND | ND | ND | 2.8 | 2.8 | 2.8 | 2.7 | 2.8 | 2.8 | 2.7 | 2.6 |
| Median Particle | 266 | ND | ND | ND | 267 | 279 | 279 | 301 | 274 | 271 | 281 | 278 |
| Size (nm) | | | | | | | | | | | | |
| Assay (mg/mL) | 0.89 | 0.89 | 0.89 | 0.85 | 0.86 | 0.80 | 0.88 | 0.88 | 0.87 | 0.86 | 0.87 | 0.87 |
| Liothyronine (% w/w) | 0.01 | 0.02 | 0.06 | 0.10 | 0.01 | 0.02 | 0.02 | 0.01 | 0.03 | 0.02 | 0.04 | 0.05 |
| Total | 0.35 | 0.39 | 0.62 | 1.19 | 0.42 | 0.42 | 0.51 | 0.40 | 0.51 | 0.51 | 0.66 | 0.64 |
| Impurities (% w/w) | | | | | | | | | | | | |
| | Formulation 12B | | | | | | | | | | | |
| Description | WS | WS | WS | WS | WS | WS | WS | WS | WS | WS | WS | WS |
| Viscosity (cps) | 1.1 | — | — | — | 1.1 | 1.2 | 1.1 | 1.1 | 1.0 | 1.1 | 1.1 | 1.2 |
| Median Particle | 2.3 | — | — | — | 2.3 | 1.7 | 1.6 | 1.7 | 2.0 | 1.8 | 1.9 | 2.1 |
| Size (μm) | | | | | | | | | | | | |
| Assay (mg/mL) | 0.90 | 0.86 | 0.81 | 0.78 | 0.79 | 0.85 | 0.82 | 0.82 | 0.77 | 0.85 | 0.81 | 0.81 |
| Liothyronine (% w/w) | 0.01 | 0.02 | 0.07 | 0.14 | 0.01 | 0.02 | 0.02 | 0.02 | 0.02 | 0.04 | 0.05 | 0.08 |
| Total | 0.36 | 0.41 | 0.77 | 1.32 | 0.38 | 0.40 | 0.48 | 0.46 | 0.49 | 0.61 | 0.63 | 0.64 |
| Impurities (% w/w) | | | | | | | | | | | | |

WS = White Suspension;
ND = Not Determined;
M = Month(s)

### Example 3: Generalized Procedure for the Preparation of Sterile Levothyroxine Sodium Aqueous Suspensions by Precipitation Followed by Particle Size Reduction

a) Levothyroxine sodium was dissolved in a non-aqueous liquid (such as, e.g., dimethyl sulfoxide (DMSO)) to obtain a solution. The solution was sterile filtered through a 0.2 or 0.22 micron filter.
b) Optionally, one or more pharmaceutically acceptable excipients (such as, e.g., surfactants, tonicity adjusting agents, buffering agents, and/or stabilizers), were added to the sterile water for injection (WFI) at 20-25° C. and stirred until dissolution. The WFI or the excipient solution was sterile filtered through a 0.2 or 0.22 micron filter.
c) The sterile levothyroxine sodium solution obtained in step a) was added into the sterile WFI or excipient solution obtained in step b) with stirring to obtain a levothyroxine sodium slurry. Optionally, additional pharmaceutically acceptable excipients (such as, e.g., surfactants, tonicity adjusting agents, buffering agents, suspending agents, viscosity enhancing agents, and/or stabilizers) were added to the API slurry.
d) The API slurry obtained in step c) was optionally subjected to particle size reduction using a high-pressure homogenizer, high shear, mechanical homogenizer or ultrasonic homogenizer.
e) Optionally, a separate excipient solution with any additional pharmaceutically acceptable excipients (such as, e.g., surfactants, tonicity adjusting agents, buffering agents, stabilizers, suspending agents, viscosity enhancing agents and/or pH adjustors) was prepared by dissolving them in sterile WFI and sterile filtering through a 0.2 or 0.22 micron filter.
f) The API suspension obtained in step e) was added to the excipient solution obtained in step (d)
g) Optionally, the pH of the suspension in step f) was adjusted to the desired target pH using sterile pH adjusting solutions that was prepared by dissolving the pH adjusting agents in sterile WFI and sterile filtering through a 0.2 or 0.22 micron
h) The final volume was made up with sterile WFI or sterile excipient solution and the final suspension composition was filled into vials or prefilled syringes, stoppered and sealed.
i) Optionally, the filled vials or prefilled syringes may be lyophilized and/or terminally sterilized.

Table 5 summarizes different exemplary formulations of aqueous levothyroxine sodium suspensions prepared by the generalized procedure outlined previously in Example 3. For the formulations disclosed in Table 5, a white to off-white aqueous suspension of levothyroxine sodium was observed.

TABLE 5
Exemplary Sterile Levothyroxine Sodium Aqueous Suspensions Prepared by Precipitation followed
by Particle Size Reduction Using GEA PANDA PLUS 2000 High Pressure Homogenizer

| Formulation# | 13A | 13B | 13C | 14A | 14B | 15 | 16 |
|---|---|---|---|---|---|---|---|
| Levothyroxine sodium (mg) | 1 | 1 | 1 | 1 | 1 | 1 | 1 |
| Dimethyl sulfoxide (mg) | 5 | 5 | 5 | 5 | 5 | 5 | 5 |
| Polysorbate-80 (mg) | 0.5 | 0.5 | 0.5 | 0.5 | 0.5 | 0.5 | 0.5 |
| Citric acid monohydrate (mg) | 4.6 | 4.6 | 4.6 | 0.4 | 0.4 | — | — |
| Tri sodium citrate dihydrate (mg) | 30.6 | 30.6 | 30.6 | 3.1 | 3.1 | — | — |
| Monobasic Sodium Phosphate Mono | — | — | — | — | — | 0.7 | 0.7 |
| Hydrate (mg) | | | | | | | |
| Dibasic sodium Phosphate anhydrous | — | — | — | — | — | — | 1 |
| (mg) | | | | | | | |
| Sodium chloride (mg) | 3 | 3 | 3 | 5 | 5 | 5 | 5 |
| Ethylenediaminetetraacetic acid (mg) | 0.2 | 0.2 | 0.2 | 0.2 | 0.2 | 0.2 | 0.2 |
| Sodium carboxymethyl cellulose (mg) | 5 | — | — | 5 | — | 5 | — |
| Povidone (mg) | — | 5 | — | — | — | — | — |
| Monothioglycerol (mg) | — | — | 5 | — | — | — | — |
| Polyethylene glycol 3350 (mg) | — | — | — | — | 5 | — | 5 |
| Water | Qs to | Qs to | Qs to | Qs to | Qs to | Qs to | Qs to |
| | 1 mL | 1 mL | 1 mL | 1 mL | 1 mL | 1 mL | 1 mL |
| Hydrochloric acid | — | — | — | Qs to | Qs to | Qs to | Qs to |
| NaOH | — | — | — | pH | pH | pH | pH |

Attributes

| | | | | | | | |
|---|---|---|---|---|---|---|---|
| Description | WS | WS | WS | WS | WS | WS | WS |
| pH | 6.08 | 6.1 | 6.1 | 6.1 | 6.2 | 7.5 | 7.5 |
| Osmolality (mOsmol/kg) | 498 | 486 | 526 | 278 | 287 | 273 | 280 |
| Viscosity (cP) | 3.3 | 1.1 | 1.2 | 3.2 | 1.3 | 3.2 | 1.4 |
| Median Particle Size (nm) | 227 | 382 | 1020 | 293 | 1633 | 570 | 2056 |
| Assay (mg/mL) | 0.92 | 0.92 | 0.92 | 0.86 | 0.86 | 0.98 | 0.96 |
| Liothyronine (% w/w) | 0.01 | 0.01 | 0.02 | 0.02 | 0.02 | 0.02 | 0.02 |
| Total Impurities (% w/w) | 0.38 | 0.40 | 0.43 | 0.65 | 0.72 | 0.72 | 0.71 |

WS = White Suspension;
QS = Quantity Sufficient

Some of the exemplary aqueous suspension formulations prepared as per the generalized procedure outlined previously in Example 3 were stored in sealed containers (Formulation 13A-13C were filled in a 2 mL USP Type I glass vial, stoppered using 13 mm Omniflex stopper FM457/0 from Datwyler and sealed; Formulation 14A and 15 were filled in a prefilled syringe ((Hypak SCF 1 MLL 27G X ½ inch needle-5B P136 from Becton Dickinson) and stoppered using Hypak BSCF 1 MLL PH701/50CBLK stopper from Becton Dickinson) and placed on stability at 60° C. as well as at ICH accelerated (40±2° C./75±5% RH) and long-term (25±2° C./60±5% RH) stability conditions. The following attributes were tested: Description, Viscosity, Assay (of levothyroxine sodium), Impurities, and Particle Size. Table 6(a) and Table 6(b) summarizes the stability data for these formulations. The data shows that the formulations are chemically stable after 1 month at 60° C. and after 6 months at either 40° C. or 25° C. Particularly surprising again is that the levels of liothyronine, the primary degradant in the liquid state, is very low even in the absence of sodium iodide often used to stabilize solution formulations of levothyroxine from deiodination. Further, the particle size of the suspensions of Formulation 13A, 14A and 15 remained stable after 6 months at either 40° C. or 25° C.

TABLE 6(a)
Consolidated Stability Data for Formulations 13A-13C

| | Stability Condition | | | | | | | | | | |
|---|---|---|---|---|---|---|---|---|---|---|---|
| | Initial | 60° C. | | | 25° C/60% RH | | | 40° C/75% RH | | | |
| | Stability Duration | | | | | | | | | | |
| Attributes | N/A | 2 D | 15 D | 1 M | 1 M | 3 M | 6 M | 1 M | 2 M | 3 M | 6 M |
| | Formulation 13A | | | | | | | | | | |
| Description | WS | WS | WS | WS | WS | WS | WS | WS | WS | WS | WS |
| Viscosity (cps) | 3.3 | ND | ND | ND | 3.1 | 2.6 | 2.4 | 3.1 | 3.0 | 2.5 | 2.3 |
| Median Particle | 227 | ND | ND | ND | 332 | 280 | 232 | 328 | 317 | 365 | 346 |
| Size (nm) | | | | | | | | | | | |
| Assay (mg/mL) | 0.92 | 0.91 | 0.88 | 0.85 | 0.91 | 0.91 | 0.90 | 0.92 | 0.90 | 0.93 | 0.90 |
| Liothyronine (% w/w) | 0.01 | 0.02 | 0.02 | 0.08 | 0.02 | 0.02 | 0.03 | 0.03 | 0.03 | 0.03 | 0.07 |
| Total | 0.38 | 0.42 | 0.83 | 1.55 | 0.48 | 0.88 | 0.64 | 0.50 | 0.58 | 1.18 | 1.17 |
| Impurities (% w/w) | | | | | | | | | | | |
| | Formulation 13B | | | | | | | | | | |
| Description | WS | WS | WS | WS | WS | WS | WS | WS | WS | WS | WS |
| Viscosity (cps) | 1.2 | ND | ND | ND | 1.1 | 1.2 | 1.2 | 1.1 | 1.3 | 1.2 | 1.2 |
| Median Particle | 0.4 | ND | ND | ND | 0.85 | 0.7 | 1 | 0.85 | 1.3 | 1 | 1.2 |
| Size (μm) | | | | | | | | | | | |
| Assay (mg/mL) | 0.92 | 0.89 | 0.91 | 0.78 | 0.90 | 0.91 | 0.89 | 0.88 | 0.93 | 0.89 | 0.89 |
| Liothyronine (% w/w) | 0.01 | 0.03 | 0.04 | 0.11 | 0.02 | 0.02 | 0.03 | 0.03 | 0.04 | 0.04 | 0.07 |
| Total | 0.40 | 0.40 | 0.68 | 1.66 | 0.48 | 0.75 | 0.84 | 0.69 | 0.85 | 1.15 | 1.36 |
| Impurities (% w/w) | | | | | | | | | | | |
| | Formulation 13C | | | | | | | | | | |
| Description | WS | WS | WS | WS | WS | WS | WS | WS | WS | WS | WS |
| Viscosity (cps) | 1.2 | ND | ND | ND | 1.2 | 1.2 | 1.2 | 1.1 | 1.2 | 1.2 | 1.2 |
| Median Particle | 1.0 | ND | ND | ND | 2.0 | 1.1 | 1.6 | 2.5 | 2.2 | 1.2 | 1.1 |
| Size (μm) | | | | | | | | | | | |
| Assay (mg/mL) | 0.92 | 0.90 | 0.86 | 0.79 | 0.88 | 0.93 | 0.87 | 0.87 | 0.88 | 0.87 | 0.84 |
| Liothyronine (% w/w) | 0.02 | 0.02 | 0.10 | 0.09 | 0.02 | 0.03 | 0.03 | 0.04 | 0.05 | 0.04 | 0.08 |
| Total | 0.43 | 0.49 | 0.68 | 0.90 | 0.50 | 0.97 | 0.80 | 0.63 | 0.72 | 0.93 | 1.12 |
| Impurities (% w/w) | | | | | | | | | | | |

D: Day(s);
M: Month(s);
WS: white suspension;
ND: Not done

TABLE 6(b)
Consolidated Stability Data for Formulations 14A and 15

| | Stability Condition | | | | | | | | | |
|---|---|---|---|---|---|---|---|---|---|---|
| | Initial | 25° C./60% RH | | | | | 40° C./75% RH | | | |
| | Stability Duration | | | | | | | | | |
| Attributes | N/A | 1 M | 2 M | 3 M | 6 M | 12 M | 1 M | 2 M | 3 M | 6 M |
| | Formulation 14A | | | | | | | | | |
| Description | WS | WS | WS | WS | WS | WS | WS | WS | WS | WS |
| Viscosity (cps) | 3.2 | 3.2 | 3.0 | 3.2 | 2.5 | 2.5 | 3.3 | 3.0 | 2.9 | 3.0 |
| Median Particle Size (nm) | 291 | 236 | 250 | 235 | 248 | 215 | 227 | 243 | 274 | 247 |
| Assay (mg/mL) | 0.86 | 0.83 | 0.85 | 0.85 | 0.84 | 0.85 | 0.85 | 0.80 | 0.83 | 0.85 |
| Liothyronine (% w/w) | 0.02 | 0.01 | 0.02 | 0.02 | 0.02 | 0.03 | 0.02 | 0.03 | 0.04 | 0.05 |
| Total Impurities (% w/w) | 0.43 | 0.52 | 0.52 | 0.53 | 0.51 | 0.54 | 0.53 | 0.61 | 0.65 | 0.80 |
| | Formulation 15 | | | | | | | | | |
| Description | WS | WS | WS | WS | WS | WS | WS | WS | WS | WS |
| Viscosity (cps) | 3.2 | 3.3 | 3.3 | 3.1 | 3.1 | 3.1 | 3.2 | 3.2 | 3.2 | 2.9 |
| Median Particle Size (nm) | 570 | 660 | 708 | 669 | 821 | 768 | 627 | 697 | 678 | 661 |
| Assay (mg/mL) | 0.98 | 0.98 | 1.00 | 0.93 | 0.90 | 0.95 | 0.96 | 0.91 | 0.91 | 0.88 |
| Liothyronine (% w/w) | 0.02 | 0.03 | 0.04 | 0.05 | 0.06 | 0.06 | 0.06 | 0.07 | 0.08 | 0.08 |
| Total Impurities (% w/w) | 0.52 | 0.83 | 0.96 | 1.17 | 1.52 | 2.09 | 1.12 | 1.54 | 2.15 | 3.43 |

M = Month(s);
WS = White Suspension

As shown in Table 6(b), the formulation having pH 6 with citrate buffer exhibits no more than about 0.4% increase in total impurities from the initial time point to 6 months at 40° C. The formulation having pH 7.5 in phosphate buffer exhibits about 3% increase in total impurities from the initial time point to 6 months at 40° C. Similarly, Formulation 14A shows an increase of not more than 0.2% increase in total impurities after 12 months at 25° C. while Formulation 15 shows an increase of about 1.5% under the same conditions. From this data, it is evident that the citrate buffered levothyroxine sodium suspension formulation having a lower pH (for example about 6) exhibits better stability as compared to the phosphate buffered composition at the higher pH (for example pH 7.5).

The impact of different concentrations of surfactant (e.g. polysorbate 80) on the efficiency of particle size reduction during the high pressure homogenization step was also evaluated, particularly to prepare formulations having lower median particle sizes. Levothyroxine sodium suspension formulations provided in Table 7(a) were prepared without a surfactant and with different concentrations of a surfactant (polysorbate 80). If used, the surfactant was added to levothyroxine sodium slurry and then the slurry was subjected to particle size reduction by milling using Microfluidics Microfluidizer M-110P as outlined previously in the general manufacturing process of Example 3.

TABLE 7a
Levothyroxine Compositions with Different Polysorbate 80 Concentrations

| Formulation# | 17 | 18 | 19 | 20 |
|---|---|---|---|---|
| Levothyroxine sodium (mg) | 1.0 | 1.0 | 1.0 | 1.0 |
| Dimethyl sulfoxide (mg) | 5.0 | 5.0 | 5.0 | 5.0 |
| Polysorbate-80 (mg) | — | 0.5 | 1.0 | 3.0 |
| Monobasic sodium phosphate | 0.345 | 0.699 | 0.699 | 0.699 |
| monohydrate (mg) | | | | |
| Dibasic sodium phosphate | 0.492 | 0.245 | 0.245 | 0.245 |
| anhydrous (mg) | | | | |
| Water for injection | Qs to 1 mL | Qs to 1 mL | Qs to 1 mL | Qs to 1 mL |

Attributes

| | | | | | | | | |
|---|---|---|---|---|---|---|---|---|
| Median Particle Size Pre-milling | 7.0 | μm | 53 | μm | 69.6 | μm | 51 | μm |
| (Pre- size reduction) | | | | | | | | |
| Median Particle Size Post-milling | 2.57 | μm | 119 | nm | 85 | nm | 88.6 | nm |

(Post- size reduction)

QS: Quantity Sufficient

The data in Table 7(a) show that addition of a surfactant significantly improves the efficiency of particle size reduction by milling. As the polysorbate concentration increases, the median particle size achieved during milling was reduced to less than 100 nm up to a concentration of 1 mg/mL; at higher concentration of polysorbate 80 (3 mg/mL), no further improvement was seen in particle size of the formulation.

Furthermore, impact of different grades of polysorbate (polysorbate 20 vs. polysorbate 80) and different concentrations of surfactant (polysorbate 80) on the stability were evaluated. Table 7(b) summarizes different exemplary formulations of aqueous levothyroxine sodium suspensions containing various grades of polysorbate were prepared by the generalized procedure as outlined previously in Example 3 (using the GEA PANDA PLUS 2000 high pressure homogenizer).

TABLE 7(b)
Sterile Levothyroxine Sodium Aqueous Suspensions with
Different Polysorbate Grades and Concentration

| Formulation# | 21 | 22 | 23 | 24 | 25 | 26 | 27 |
|---|---|---|---|---|---|---|---|
| Levothyroxine sodium (mg) | 1 | 1 | 1 | 1 | 1 | 1 | 1 |
| Dimethyl sulfoxide (mg) | 5 | 5 | 5 | 5 | 5 | 5 | 5 |
| Polysorbate-80 (mg) | 0.5 | 0.5 | 0.5 | 3 | 3 | 3 | — |
| Polysorbate-20 (mg) | — | — | — | — | — | — | 0.5 |
| Citric acid monohydrate (mg) | — | — | — | — | — | — | 0.42 |
| Tri sodium citrate dihydrate (mg) | — | — | — | — | — | — | 3.06 |
| Monobasic Sodium Phosphate Mono | 3.25 | 3.25 | 3.25 | 3.25 | 3.25 | 3.25 | — |
| Hydrate (mg) | | | | | | | |
| Dibasic sodium Phosphate anhydrous | 0.245 | 0.245 | 0.245 | 0.245 | 0.245 | 0.245 | — |
| (mg) | | | | | | | |
| Sodium chloride (mg) | — | — | 5 | — | — | 5 | 5 |
| Ethylenediaminetetraacetic acid (mg) | — | — | 0.2 | — | — | 0.2 | 0.2 |
| Sodium carboxymethyl cellulose | — | — | 5 | — | — | 5 | — |
| (mg) | | | | | | | |
| Mannitol (mg) | — | 30 | — | — | 30 | — | — |
| Glycerol (mg) | 16 | — | — | 16 | — | — | — |
| Water | Qs to | Qs to | Qs to | Qs to | Qs to | Qs to | Qs to |
| | 1 mL | 1 mL | 1 mL | 1 mL | 1 mL | 1 mL | 1 mL |

Attributes

| | | | | | | | |
|---|---|---|---|---|---|---|---|
| Description | WS | WS | WS | WS | WS | WS | WS |
| pH | 6.18 | 6.12 | 5.82 | 6.26 | 6.14 | 6.04 | 6.1 |
| Osmolality (mOsmol/kg) | — | — | 289 | — | — | 327 | 290 |
| Viscosity (cP) | — | — | 2.8 | — | — | 2.7 | 2.9 |
| Median Particle Size (μm) | 1.2 | 2.4 | 0.25 | 1.6 | 1.4 | 0.31 | 0.32 |
| Assay (mg/mL) | 0.94 | 0.93 | 0.92 | 0.95 | 0.97 | 1.11 | 0.96 |
| Liothyronine (% w/w) | 0.05 | 0.05 | 0.05 | 0.05 | 0.05 | 0.05 | 0.05 |
| Total Impurities (% w/w) | 0.42 | 0.43 | 0.44 | 0.52 | 0.58 | 0.45 | 0.33 |

WS: White Suspension;
QS: Quantity Sufficient

The exemplary aqueous suspension formulations prepared as per the generalized procedure outlined previously in Example 3 were stored in a prefilled syringe (Formulation 21-23 and 27 were filled in Hypak SCF 1 MLL 27G X ½ inch needle-5B P136 from Becton Dickinson) and stoppered using Hypak BSCF 1 MLL PH701/50CBLK stopper from Becton Dickinson; Formulation 26 was filled in Novo Ompi EZ-FILL 7600007.6903 and stoppered using Hypak BSCF 1 MLL PH701/50CBLK stopper from Becton Dickinson) and placed on stability at ICH accelerated (40±2° C./75±5% RH), long-term (25±2° C./60±5% RH) and refrigerated 2-8° C. stability conditions. The following attributes were tested: Description, Viscosity, Assay (of levothyroxine sodium), Impurities, and Particle Size. Table 7(c)-summarize the stability data for these formulations.

The formulation 23 containing 0.5 mg/mL polysorbate 80 exhibits no more than about 0.3% increase in total impurities from the initial time point to 2 months at 40° C. Similarly, the formulation 27 containing 0.5 mg/mL polysorbate 20 exhibits no more than about 0.3% increase in total impurities at the same conditions. As far as the physical stability, the particle size distribution of both the formulations are stable.

Therefore, no difference in physical and chemical stability was observed for polysorbate 20 versus polysorbate 80.

Formulation 23 containing 0.5 mg/mL of polysorbate 80 exhibits no more than about 0.3% increase in total impurities from the initial time point to 2 months at 40° C. However, the formulation 26 containing 3 mg/mL of polysorbate 80 exhibits about 1.6% increase in total impurities at the same conditions. Therefore, a lower concentration of polysorbate 80 provides more stable product.

Formulation 23 containing ethylenediaminetetraacetic acid (EDTA), a stabilizer, exhibits no more than about 0.3% increase in total impurities from the initial time point to 2 months at 40° C., as provided in Table 7(c). However, the formulations 21 and 22 manufactured without EDTA exhibits about 0.6% increase in total impurities at the same condition, as provided in Table 7(c). From the data, it is evident that the formulation containing stabilizer (for example EDTA) is more stable than the one containing no stabilizer.

TABLE 7(c)
Consolidated Stability Data for the Formulations 26, 21-23 and 27

| | Stability Condition | | | | | | |
|---|---|---|---|---|---|---|---|
| | Initial | 2-8° C. | | 25° C./60% RH | | 40° C./75% RH | |
| | Stability Duration | | | | | | |
| Attributes | N/A | 1 M | 2 M | 1 M | 2 M | 1 M | 2 M |
| | Formulation 26 | | | | | | |
| Description | WS | WS | WS | WS | WS | WS | WS |
| Viscosity (cps) | 2.7 | — | 2.61 | — | 2.56 | — | 2.61 |
| pH | 6.04 | 5.91 | 6.03 | 5.94 | 6.08 | 5.93 | 5.91 |
| Median Particle Size (nm) | 311 | 290 | 353 | 310 | 355 | 303 | 351 |
| Assay (mg/mL) | 1.11 | 1.06 | 1.10 | 1.09 | 1.09 | 1.07 | 1.04 |
| Liothyronine (% w/w) | 0.05 | 0.05 | 0.05 | 0.05 | 0.06 | 0.08 | 0.12 |
| Total Impurities (% w/w) | 0.45 | 0.49 | 0.57 | 0.65 | 0.89 | 1.41 | 2.12 |
| | Formulation 21 | | | | | | |
| Description | WS | WS | WS | WS | WS | WS | WS |
| Viscosity (cps) | — | — | — | — | — | — | — |
| pH | 6.18 | 6.22 | — | 6.20 | — | 6.23 | — |
| Median Particle Size (μm) | 1.2 | — | — | — | — | — | — |
| Assay (mg/mL) | 0.94 | 0.93 | — | 0.94 | — | 0.91 | — |
| Liothyronine (% w/w) | 0.05 | 0.05 | 0.05 | 0.05 | 0.05 | 0.06 | 0.07 |
| Total Impurities (% w/w) | 0.42 | 0.39 | 0.48 | 0.44 | 0.63 | 0.66 | 1.15 |
| | Formulation 22 | | | | | | |
| Description | WS | WS | WS | WS | WS | WS | WS |
| Viscosity (cps) | — | — | — | — | — | — | — |
| pH | 6.12 | 6.16 | — | 6.16 | — | 6.15 | — |
| Median Particle Size (μm) | 2.4 | — | — | — | — | — | — |
| Assay (mg/mL) | 0.93 | 0.92 | — | 0.92 | — | 0.91 | — |
| Liothyronine (% w/w) | 0.05 | 0.05 | 0.05 | 0.05 | 0.05 | 0.06 | 0.07 |
| Total Impurities (% w/w) | 0.43 | 0.39 | 0.47 | 0.42 | 0.53 | 0.59 | 1.0 |
| | Formulation 23 | | | | | | |
| Description | WS | WS | WS | WS | WS | WS | WS |
| Viscosity (cps) | 2.8 | — | — | — | — | — | — |
| pH | 5.8 | 5.9 | 5.94 | 5.9 | 5.91 | 5.9 | 5.92 |
| Median Particle Size (nm) | 254 | 238 | 231.7 | 231 | 242 | 232 | 234.3 |
| Assay (mg/mL) | 0.92 | 0.91 | 0.90 | 0.9 | 0.87 | 0.91 | 0.96 |
| Liothyronine (% w/w) | 0.05 | 0.05 | 0.05 | 0.06 | 0.05 | 0.06 | 0.06 |
| Total Impurities (% w/w) | 0.44 | 0.38 | 0.45 | 0.40 | 0.52 | 0.45 | 0.68 |
| | Formulation 27 | | | | | | |
| Description | WS | WS | WS | WS | WS | WS | WS |
| Viscosity (cps) | 2.9 | — | 3.0 | — | 3.0 | — | 2.9 |
| pH | 6.10 | 6.07 | 6.08 | 6.10 | 6.10 | 6.10 | 6.10 |
| Median Particle Size (nm) | 318 | 283 | 275 | 249 | 253 | 248 | 244 |
| Assay (mg/mL) | 0.95 | 0.94 | 0.98 | 0.95 | 0.97 | 0.94 | 0.98 |
| Liothyronine (% w/w) | 0.05 | 0.05 | 0.05 | 0.05 | 0.05 | 0.05 | 0.06 |
| Total Impurities (% w/w) | 0.32 | 0.41 | 0.35 | 0.42 | 0.39 | 0.53 | 0.63 |

WS: White Suspension;
M: Month(s)

### Example 4

### Generalized Procedure for the Preparation of Sterile Levothyroxine Sodium Aqueous Suspensions by Precipitation and Particle Size Reduction Followed by Sterile Filtration

a) Levothyroxine sodium was dissolved in a non-aqueous liquid (such as, e.g., dimethyl sulfoxide (DMSO)) to obtain a solution.
b) Optionally, one or more pharmaceutically acceptable excipients (such as, e.g., surfactants, tonicity adjusting agents, buffering agents, and/or stabilizers), were added to the sterile water for injection (WFI) at 20-25° C. and stirred until dissolution.
c) The sterile levothyroxine sodium solution obtained in step a) was added into the sterile WFI or excipient solution obtained in step b) with stirring to obtain a levothyroxine sodium slurry. Optionally, additional pharmaceutically acceptable excipients (such as, e.g., surfactants, tonicity adjusting agents, buffering agents, and/or stabilizers) were added to the API slurry.
d) Optionally, the pH of the suspension in step c) was adjusted to the desired target pH using pH adjusting solutions.
e) The final volume was made up with sterile WFI or sterile excipient solution.
f) The suspension obtained in step e) was optionally subjected to particle size reduction using a high-pressure homogenizer, high shear, mechanical homogenizer or ultrasonic homogenizer
g) The suspension obtained in step f) was filtered through a 0.2 or 0.22 micron sterile filter.
h) The final suspension composition was filled into vials or prefilled syringes, stoppered and sealed. Optionally, the filled vials or prefilled syringes may be lyophilized and/or terminally sterilized.

Table 8(a) summarizes different exemplary formulations of aqueous levothyroxine sodium suspensions prepared by the generalized procedure as outlined previously in Example 4. For the formulations disclosed in Table 9(a), a white to off-white aqueous suspension of levothyroxine sodium was observed.

TABLE 8 a
Exemplary Sterile Levothyroxine Sodium Aqueous
Suspensions Prepared by Precipitation, Particle
Size Reduction by Noozle Nano Microfluidization
Homogenizer Followed by Sterile Filtration

| Formulation # | 28 | 29 |
|---|---|---|
| Levothyroxine sodium (mg) | 1.0 | 1.0 |
| Dimethyl sulfoxide (mg) | 5.0 | 5.0 |
| Poly sorbate-80 (mg) | 3.0 | 3.0 |
| Monobasic sodium phosphate | 0.699 | 0.699 |
| monohydrate (mg) | | |
| Dibasic sodium phosphate anhydrous (mg) | 0.245 | 0.245 |
| Glycerol (mg) | 16.0 | — |
| Mannitol (mg) | — | 30.0 |
| Water | Qs to 1 mL | Qs to 1 mL |
| NaOH | — | Qs to |
| | | adjust pH |
| pH | 6.83 | 6.70 |
| Osmolality (mOsmol/kg) | 260 | 258 |

Attributes

| | White | White |
|---|---|---|
| Description | Suspension | Suspension |
| Median Particle Size (nm) | 91.4 | 100.3 |
| Assay (mg/mL) | 0.98 | 0.99 |
| Liothyronine (% w/w) | 0.06 | 0.06 |
| Total Impurities (% w/w) | 1.06 | 1.32 |

QS: Quantity Sufficient

The exemplary aqueous suspension formulation 28 and 29 were prepared at 1 mg/ml concentration and particle size was reduced by milling using high pressure homogenizer followed by filtered through 0.2 or 0.22 micron sterile filter. This indicates that the sterile filtration can be performed for the suspensions with a median particle size of about 100 nm according to the present invention.

### Example 5: Dissolution Profile of Levothyroxine Sodium Suspension

Levothyroxine sodium suspension formulations according to the present invention were evaluated for their drug release profile. The below dissolution data demonstrates that the formulations are sustained release formulations. Further, levothyroxine sodium suspension composition according to the present invention have broad range of drug release profile and suitable for drug release over a prolonged period, for example up to six months. Specifically, the suspension is suitable for administration once in two days, twice weekly, once weekly, twice monthly, once monthly, once every two months, or 3 months, or 6 months by intravenous, intramuscular or subcutaneous administration.

### Dissolution Test Using USP Type-II Apparatus:

### a) Dissolution Profile at pH 7.4 in Scissors Buffer

Dissolution experiments were performed on a levothyroxine sodium suspension of this invention (Formulation 15 at 1 mg/mL) and a levothyroxine sodium solution formulation (30) using USP Type-II in Scissors buffer at a pH of 7.4. Scissors buffer was prepared by dissolving 6.4 g Sodium Chloride, 0.09 g Magnesium Chloride Hexahydrate, 0.4 g Potassium Chloride, 0.2 g Calcium Chloride and 2.1 g Sodium bi carbonate in 1000 mL of water and pH of the solution was adjusted to 7.4 using diluted hydrochloric acid.

A Levothyroxine sodium lyophilized powder formulation currently marketed for intravenous administration containing 500 mcg of levothyroxine sodium, 0.944 mg dibasic sodium phosphate heptahydrate, 3.0 mg mannitol and sodium hydroxide (QS to pH) in single-use amber glass vial was reconstituted with 5 ml of 0.9% sodium chloride to yield a 100 mcg/mL solution (Formulation 30).

Dissolution Procedure: 900 mL of dissolution media (pH 7.4 Scissors Buffer) was added into 1000 mL dissolution bowls and the temperature was maintained at 37° C. About 0.5 mL of the levothyroxine suspension or the solution formulation was packed in 1000 kda molecular weight cut-off (MWCO) dialysis membrane. The sample packed in the membrane was placed into the basket and stirred at 50 RPM. 5 mL sample was collected at different timepoints by means of autosampler and analysed for levothyroxine content by HPLC.

The drug release from levothyroxine sodium suspension formulation 15 (d50: 708 nm) and levothyroxine sodium solution formulation was about 2% in 0.5 hour, about 14% in 8 hours, and about 26% in 14 hours. In comparison, the drug release from the solution formulation is about 40% in 0.5 hour, 82% in 8 hours, with complete release (104%) in 14 hours. Thus, a prolonged drug release profile is demonstrated for the suspension formulation compared to a marketed solution product.

### b) Dissolution Profile at pH 8.0 in Phosphate Buffer

Dissolution experiments were performed on levothyroxine sodium suspensions (12A and 13A) and a levothyroxine sodium solution formulation using USP Type-II in phosphate buffer at a pH of 8.0. Levothyroxine lyophilized powder 500 mcg/vial, as mentioned above, was reconstituted with 5 ml of 0.9% sodium chloride to obtain 100 mcg/mL (Formulation 30). The phosphate buffer was prepared by dissolving 14.2 g of disodium hydrogen phosphate in 1000 mL of water and the pH was adjusted to 8.0 with dilute Hydrochloric acid.

Dissolution Procedure: 900 mL of dissolution media (pH 8.0 Di Sodium Hydrogen Phosphate Buffer) was added into 1000 mL dissolution bowls and the temperature was maintained at 37° C. About 0.5 mL of the levothyroxine suspension or the solution formulation was packed in 1000 kda molecular weight cut-off (MWCO) dialysis membrane. The sample packed in the membrane was placed into the basket and stirred at 50 RPM. 5 mL sample was collected at different timepoints by means of autosampler and analysed for levothyroxine content by HPLC.

The drug release from both levothyroxine sodium suspension Formulation 12A (d50: 279 nm) and Formulation 13A (d50: 280 nm) was about 3% in 2 hours, and about 20% in 14 hours. In comparison, the drug release from the solution formulation is about 72% in 2 hours and 91% in 14 hours. Thus, a prolonged drug release profile is demonstrated for the suspension formulation compared to a marketed solution product.

The levothyroxine sodium suspension composition according to the present invention has broad range of drug release profile suitable for sustained release over a prolonged period, for example up to six months. Specifically, the suspension is suitable for once in two days, twice weekly, once weekly, twice monthly, once monthly, etc administration by intravenous, intramuscular or subcutaneous.

It is to be understood that while the invention has been described in conjunction with the preferred specific embodiments thereof, that the foregoing description and the examples that follow are intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains. In addition to the embodiments described herein, the present disclosure contemplates and claims those inventions resulting from the combination of features of the invention cited herein and those of the cited prior art references which complement the features of the present invention. Similarly, it will be appreciated that any described material, feature, or article may be used in combination with any other material, feature, or article, and such combinations are considered within the scope of this invention.

1. A long-acting parenteral pharmaceutical dispersion comprising (a) a therapeutically effective amount of levothyroxine or a pharmaceutically acceptable salt thereof, wherein the levothyroxine or its pharmaceutically acceptable salt is primarily dispersed in a pharmaceutically acceptable liquid carrier and (b) one or more pharmaceutically acceptable excipients.

2. The pharmaceutical dispersion according to claim 1, wherein at least 60% by weight of the levothyroxine or its pharmaceutically acceptable salt in the pharmaceutical dispersion is in particulate form.

3. The pharmaceutical dispersion according to claim 1, wherein at least 70% by weight of the levothyroxine or its pharmaceutically acceptable salt in the pharmaceutical dispersion is in particulate form.

4. The pharmaceutical dispersion according to claim 1, wherein the pharmaceutical dispersion provides sustained-release or extended-release of levothyroxine or its pharmaceutically acceptable salt.

5. The pharmaceutical dispersion according to claim 1, wherein the pharmaceutical dispersion releases a therapeutically effective amount of levothyroxine or its pharmaceutically acceptable salt over a period of two days to six months.

6. The pharmaceutical dispersion according to claim 5, wherein the amount of levothyroxine or pharmaceutically acceptable salt thereof released daily from the pharmaceutical dispersion after parenteral administration is bioequivalent to the effect of once daily oral dosing of 20 mcg to 500 mcg.

7. The pharmaceutical dispersion according to claim 1, wherein the pharmaceutical dispersion comprises a sodium salt or potassium salt of levothyroxine.

8-9. (canceled)

10. The pharmaceutical dispersion according to claim 1, wherein the pharmaceutically acceptable carrier is selected from water, one or more non-aqueous liquids, or a mixture of water and one or more non-aqueous liquids.

11. The pharmaceutical dispersion according to claim 10, wherein the non-aqueous liquid is selected from ethanol, propylene glycol (PG), polyethylene glycol (PEG), acetone, glycerin, N-methylformamide, N,N-dimethylformamide (DMF), N,N-dimethylacetamide (DMA), dimethylsulfoxide (DMSO), N-methylpyrrolidone (NMP), 2-pyrrolidone, or any combination of any of the foregoing.

12. The pharmaceutical dispersion according to claim 1, wherein the pharmaceutically acceptable excipients is selected from one or more suspending agents, surfactants, antioxidants, preservatives, buffering agents, pH adjusting agents, stabilizing agents, isotonicity agents, chelating agents, viscosity enhancing agents and any combination of any of the foregoing.

13. The pharmaceutical dispersion according to claim 12, wherein the suspending agent is selected from cellulose derivatives and salts thereof, polyvinylpyrrollidone, alginates, chitosan, dextrans, gelatin, polyethylene glycols, polyoxyethylene and polyoxypropylene ethers.

14-15. (canceled)

16. The pharmaceutical composition according to claim 12, wherein the stabilizing agents is selected from sodium iodide, potassium iodide, tromethamine, ethylenediaminetetraacetic acid (EDTA) or its sodium salt, amino acids such as glycine, lysine, aspartic acid, methionine or their salts and the like, TTTA (1,7,13-triaza-4,10,16-trioxacyclooctadecane-N,N′,N″-triacetate), DOTRP (tetraethyleneglycol-1,5,9-triazacyclododecane-N,N′,N″-tris(methylene phosphonic acid), and EGTA (ethylene glycol-bis(β-aminoethyl ether)-tetraacetic acid), DOTA (1,4,7,10-tetraazacyclododecane-1,4,7,10-tetraacetic acid), tetrasodium-iminodisuccinate (IDS), tartaric acid, citric acid, acetic acid, ascorbic acid, formic acid, succinic acid, pentetic acid, tannic acid, diethylenetriamine pentaacetate (DTPA) or its salts, trisodium ethylenediamine disuccinate and the like, and combinations thereof.

17-21. (canceled)

22. The pharmaceutical dispersion according to claim 1, wherein the pH of the pharmaceutical dispersion is 4 to 8.

23-26. (canceled)

27. A pharmaceutical dispersion comprising (a) levothyroxine or its pharmaceutically acceptable salt dispersed in a mixture of water and one or more non-aqueous solvents, (b) surfactant, (c) tonicity agent, (d) one or more buffers, (e) stabilizing agent, (f) suspending agent, (g) optionally viscosity enhancing agent (h) optionally chelating agent, and (i) optionally one or more pH adjusting agents.

28. The pharmaceutical dispersion according to claim 27, wherein the dispersion comprises (a) levothyroxine or its pharmaceutically acceptable salt dispersed in a mixture of water and dimethyl sulfoxide, (b) polysorbate, (c) sodium chloride, glycerol or mannitol (d) one or more buffers, (e) EDTA or its salt, (f) carboxymethylcellulose or its salt and (g) optionally one or more pH adjusting agent.

29. The pharmaceutical dispersion according to claim 27, wherein the dispersion comprises (a) levothyroxine or its pharmaceutically acceptable salt dispersed in water, (b) polysorbate, (c) sodium chloride, glycerol, or mannitol, (d) one or more buffers, (e) EDTA or its salt, (f) carboxymethylcellulose or its salt, and (g) optionally one or more pH adjusting agents.

30. The pharmaceutical dispersion according to claim 27, wherein the dispersion comprises (a) levothyroxine or its pharmaceutically acceptable salt dispersed in water, (b) polysorbate, (c) sodium chloride, glycerol, or mannitol (d) one or more buffers, (e) EDTA or its salt, (f) povidone, and (g) optionally one or more pH adjusting agents.

31. The pharmaceutical dispersion according to claim 27, wherein the dispersion comprises (a) levothyroxine or its pharmaceutically acceptable salt dispersed in water and dimethyl sulfoxide, (b) polysorbate, (c) sodium chloride, glycerol, or mannitol (d) one or more buffers, (e) EDTA or its salt, (f) povidone, and (g) optionally one or more pH adjusting agents.

32. The pharmaceutical dispersion according to claim 27, wherein the composition comprises (a) levothyroxine or its pharmaceutically acceptable salt dispersed in water and dimethyl sulfoxide, (b) polysorbate, (c) sodium chloride, glycerol, or mannitol (d) one or more buffers, (e) EDTA or its salt, (f) monothioglycerol, and (g) optionally one or more pH adjusting agents.

33. (canceled)

34. The pharmaceutical dispersion according to claim 1, wherein the pharmaceutical dispersion has no more than about 0.5% (by HPLC) increase in the amount of liothyronine from an initial point to three months when stored at 40° C. and 75% relative humidity.

35. A method for treating myxedema coma, hypothyroidism or other thyroid related disorder in a patient in need thereof comprising administering to the patient an effective amount of the pharmaceutical dispersion of claim 1.

36-38. (canceled)

39. The method of claim 35, wherein the pharmaceutical dispersion is administered to the patient once in 2 days to once in 6 months.

40. A process for preparing sterile levothyroxine or its pharmaceutically acceptable salt comprising
a) dissolving levothyroxine or its pharmaceutically acceptable salt in a suitable liquid medium to form a solution,
b) sterilizing the solution from step (a), and
c) isolating the sterile levothyroxine or its pharmaceutically acceptable salt.

41-42. (canceled)

43. A freeze dried long-acting composition comprising a therapeutically effective amount of levothyroxine or a pharmaceutically acceptable salt thereof and one or more pharmaceutically acceptable excipients.

44. A method of preparing a pharmaceutical composition suitable for intravenous, intramuscular or subcutaneous administration comprising reconstituting the freeze dried long-acting composition of claim 43 with a diluent to obtain an aqueous dispersion.
